(19) United States
(12) Patent Application Publication
(10) Pub. No.: US 2026/0354661 A1
(43) Pub. Date: Nov. 12, 2026

(54) REAL-TIME MULTISPECTRAL POLARIMETRIC IMAGING FOR NONINVASIVE IDENTIFICATION OF NERVES AND OTHER TISSUES

(71) Applicant: University of Rochester, Rochester, NY (US)

(72) Inventors: Wayne H. KNOX, Rochester, NY (US); Haolin LIAO, Rochester, NY (US); Gregory HEYWORTH, Rochester, NY (US); David MITTEN, Rochester, NY (US)

(21) Appl. No.: 19/137,887

(22) PCT Filed: Dec. 12, 2023
(86) PCT No.: PCT/US2023/083680

§ 371 Date: Jun. 11, 2025

Related U.S. Application Data
(60) Provisional application No. 63/387,089, filed on Dec. 12, 2022.

(51) Int. Cl. A61B 1/00; A61B 1/06

(52) U.S. Cl. CPC A61B 1/000094; A61B 1/00048; A61B 1/0638; A61B 1/0646

(57) ABSTRACT

Systems and methods for automatically identifying tissues using imaging, and more particularly systems and methods using polarimetric imaging for tissue identification, in real-time. A system for tissue identification includes a light source for illuminating a region of interest with polarized light, an image sensor for acquiring images of the region of interest, at least one analyzer disposed within the field of view of the image sensor, and a processor configured to obtain a series of images of the region of interest acquired using more than one polarization angle. A tissue is identified within the region of interest based on an intensity of one or more pixels of the series of images.

### CROSS-REFERENCE TO RELATED APPLICATIONS

This application claims priority to U.S. Provisional Application No. 63/387,089, filed on Dec. 12, 2022, now pending, the disclosure of which is incorporated herein by reference.

### FIELD OF THE DISCLOSURE

The present disclosure relates to automatic tissue identification using imaging, and more particularly systems and methods using polarimetric imaging for tissue identification.

### BACKGROUND OF THE DISCLOSURE

In the field of surgery, a source of patient injury is human error. For example, different types of tissues can be difficult to distinguish by simple visual inspection. The problem has been described as being similar to digging for a buried wire that needs maintenance. A storm is coming and the job must be completed as quickly as possible. It would be beneficial to have an estimate of how deep the wire is buried and start digging quickly, but then slow down when getting close to the wire to reduce the probability of cutting it. The digging process would also likely involve hitting/avoiding materials and objects like rocks, clay, sand, roots, etc. that all have different visual and mechanical properties. In the case of surgery, it would be beneficial to have an aid to help distinguish the different materials so that a surgeon is better able to reach the target tissue while avoiding damage to other tissues.

There is an unmet need for a real-time method to aid the surgeon in differentiating tissues such as, for example, nerves, from other tissue types such as fat, muscle, tendons, cartilage, arteries, and veins.

### BRIEF SUMMARY OF THE DISCLOSURE

The present disclosure provides techniques for automatically identifying tissues and can be used in real-time tissue identification. Instead of a very limited view provided through a surgical microscope, the presently-disclosed techniques can be used on wide fields-of-view. In this way, the technique can be used for visualizing the entire surgical field in an open surgery (though it is not limited to such open surgeries). An experimental embodiment was used to examine human cadaver (human arm) and was found to enable the differentiation of nerves from other nearby tissues.

In an embodiment, a system for tissue identification includes a light source configured to illuminate a region of interest with polarized light. For example, the light may be linearly polarized. A polarization of the polarized light is configured to rotate during a sample period. For example, the polarization of the polarized light may be configured to rotate at least ¼ rotation during the sample period. In another example, the polarization of the polarized light may be configured to rotate at least one full rotation during the sample period. In another example, the polarization of the polarized light may be configured to rotate at least ½ rotation during the sample period. The system includes an image sensor for acquiring images of the region of interest illuminated by the polarized light of the light source. The image sensor has a field of view. An analyzer is disposed within the field of view of the image sensor. The analyzer has a polarization which rotates to maintain a non-zero angle (for example, 90°) relative to the polarization of the polarized light. A processor is in electronic communication with the image sensor.

The processor is configured to obtain, from the image sensor, a series of images of the region of interest acquired during at least one sample period. The processor is configured to and identify a tissue within the region of interest based on an intensity of one or more pixels of the series of images varying according to the polarization angle of the rotating polarized light. The processor may be configured to identify the tissue as nerve tissue. The processor may be configured to identify the tissue as nerve, artery vein, fat, or muscle. The processor may be configured to distinguish tissue types in the region of interest based on differences in the variability of intensity associated with tissue types (e.g., differences in a variability of intensity of muscle tissue compared to a variability of intensity of nerve tissue, etc.) The processor may be further configured to identify the tissue as abnormal tissue (e.g., diseased or physically damaged). For example, the processor may identify the tissue as abnormal based on the varying intensity. In some embodiments, the processor is further configured to determine an orientation of the identified tissue.

In some embodiments, the image sensor acquires images in one or more limited spectral bands, and the processor identifies the tissue within the region of interest based on an intensity when illuminated by the one or more limited spectral bands. For example, the light source may be configured to provide light in one or more limited spectral bands. In another example, the light source may be a broadband light source (e.g., a white light source) and an optical filter may be disposed between the light source and the image sensor. Such embodiments may be used to acquire images in one or more limited spectral bands. For example, the image sensor may acquire images in a first limited spectral band for at least a first full rotation of the polarization of the polarized light and in a second limited spectral band for at least a second full rotation of the polarization of the polarized light.

The processor may be configured to identify the tissue by determining a frequency spectra time series of images obtained from the image sensor and determining pixels corresponding to the tissue based on a peak amplitude of a frequency corresponding to the tissue. In some embodiments, the processor is configured to identify the tissue using phase-sensitive detection (lock-in signal detection) to determine pixels corresponding to the tissue. For example, the processor may be configured to remove an offset (e.g., a mean value) from a set of time series values of a pixel to generate a centered signal for the pixel, mix a reference signal with the centered signal for the pixel, shift the phase of the reference signal and/or the centered signal to determine a phase-locked signal, and calculate an average value of the phase-locked signal.

The processor may be further configured to generate an image mask based on image pixels corresponding to the identified tissue. The processor may be further configured to alter an image or a series of images (e.g., a video) using the image mask. In some embodiments, altering the image or series of images includes one or more of increasing an intensity of an unmasked portion of the image, drawing a boundary line around an unmasked portion of the image, changing a color of an unmasked portion of the image, animating an unmasked portion of the image. In some embodiments, the system further includes a projector in electronic communication with the processor, and the projector may be configured to project a highlight on the region of interest based on the identified tissue. In some embodiments, the system further includes a wearable display in electronic communication with the processor. For example, the wearable display may be configured to provide augmented reality information to a wearer. The processor may be further configured to provide a highlight overlaying the region of interest based on the identified tissue to a user using the wearable display.

In another aspect, a method for tissue identification includes illuminating a region of interest with a rotating linearly-polarized light. A series of images of the region of interest is obtained using an image sensor via an analyzer rotating synchronously with the linearly-polarized light such that the light received by the image sensor is cross polarized. A tissue is identified within the region of interest based on an intensity of one or more pixels of the series of images varying according to the polarization angle of the rotating linearly-polarized light. For example, the tissue may be identified as nerve tissue. In another example, the tissue may be identified as nerve, artery vein, fat, or muscle. The tissue may be identified by distinguishing tissue types in the region of interest based on differences in the variability of intensity associated with tissue types. The method may include identifying the tissue as abnormal tissue (e.g., diseased or physically damaged) based on the varying intensity of the tissue. The method may include determining an orientation of the identified tissue based on an intensity of the tissue corresponding to a rotational position of the rotating linearly-polarized light, and relative to the varying intensity of the tissue.

In some embodiments, the series of images is obtained in one or more limited spectral bands, and the tissue is identified based on an intensity when illuminated by the one or more limited spectral bands. For example, the illumination light may be configured to have one or more limited spectral bands. The images may be acquired in a first limited spectral band for at least a first full rotation of the polarization of the polarized light. The images may be acquired in a second limited spectral band for at least a second full rotation of the polarization of the polarized light.

In some embodiments, identifying the tissue includes calculating a frequency spectra of the time series of images obtained from the image sensor, and determining pixels corresponding to the tissue based on a peak amplitude of a frequency corresponding to the tissue. In some embodiments, identifying the tissue includes removing an offset from a set of time series values of a pixel to generate a centered signal for the pixel, mixing a reference signal with the centered signal for the pixel, shifting the phase of the reference signal and/or the centered signal to determine a phase-locked signal; and calculating an average value of the phase-locked signal.

The method may include generating an image mask based on image pixels corresponding to the identified tissue. In some embodiments, an image or a series of images (e.g., a movie) is altered using the image mask. Altering the image or series of images may include one or more of increasing an intensity of an unmasked portion of the image, drawing a boundary line around an unmasked portion of the image, changing a color of an unmasked portion of the image, and animating an unmasked portion of the image. In some embodiments, the method includes projecting a highlight on the region of interest based on the identified tissue. In some embodiments, the method includes providing augmented reality information to a wearable display. For example, the augmented reality information may be a highlight overlaying the region of interest based on the identified tissue.

In another aspect, a non-transitory computer-readable medium may have stored thereon a program for instructing a processor to perform any of the methods described herein. For example, the stored program may include instructions for a processor to: illuminate a region of interest with a rotating linearly-polarized light; obtain a series of images of the region of interest using an image sensor via an analyzer rotating synchronously with the linearly-polarized light such that the light received by the image sensor is cross polarized; and identify a tissue within the region of interest based on an intensity of one or more pixels of the series of images varying according to the polarization angle of the rotating linearly-polarized light.

In some embodiments, the stored program includes instructions to: calculate a frequency spectra of the time series of images obtained from the image sensor; and determine pixels corresponding to the tissue based on a peak amplitude of a frequency corresponding to the tissue. In some embodiments, the stored program includes instructions to: remove an offset from a set of time series values of a pixel to generate a centered signal for the pixel; mix a reference signal with the centered signal for the pixel; shift the phase of the reference signal and/or the centered signal to determine a phase-locked signal; and calculate an average value of the phase-locked signal.

In some embodiments, the stored program includes instructions to generate an image mask based on image pixels corresponding to the identified tissue. In some embodiments, the stored program includes instructions to alter an image or a series of images using the image mask.

### DESCRIPTION OF THE DRAWINGS

For a fuller understanding of the nature and objects of the disclosure, reference should be made to the following detailed description taken in conjunction with the accompanying drawings, in which:

FIG. 1 is a photograph showing four different types of tissue. (from J. Cha et al., “Real-time, label-free, intraoperative visualization of peripheral nerves and micro-vasculatures using multimodal optical imaging techniques,” Biomed Opt Express, vol. 9, no. 3, p. 1097, March 2018).

FIG. 2 is a diagram of a system according to an embodiment of the present disclosure.

FIG. 3 shows 8-bit gray results of imaging the sciatic nerve (white arrow) in ex vivo chicken thigh using: (a) traditional white LED light imaging; (b) crossed-circular polarization imaging (CPI); (c) crossed-linear polarization imaging arranged such that the polarizer was at 0 degree to the nerve; and (d) crossed-linear polarization imaging while the polarizer was arranged at 45 degrees to the nerve.

FIG. 4 shows the imaging subtraction result of subtracting FIG. 3(c) from FIG. 3(d) (cross-polarization (XP)@45°-XP@0°).

FIG. 5 is a chart showing the measured reflectance of five different tissue types using rotating crossed polarization.

FIG. 6 shows an image (frame) from a video of a chicken sciatic nerve surrounded by muscle and other tissues under a rotating cross-linearly polarized imaging (XPI) system: (a) the nerve exhibits periodic white highlighting by the XPI light having rotating polarization; and (b) the periodic highlighting signal was extracted by a processor using techniques as described herein and overlaid on the image thereby highlighting the nerve in green to serving as a visualization aid as to the location of the nerve (green in original is shown as increased brightness in FIG. 6(b)).

FIG. 7 is a set of charts showing the rotational variance of different tissues illuminated by light sources of four colors with the XPI system rotating at a constant speed: (a) 460 nm (blue); (b) 530 nm (green); (c) 590 nm (amber); and (d) 630 nm (red).

FIG. 8 is a set of charts comparing the spectral reflective intensities of different tissues in XP@0° and in XP@45°

FIG. 9 is a chart showing an angle-sensitive component versus a mean-value for various tissue types.

FIG. 10 is a chart depicting a point cloud used to display image data.

FIG. 11 is a chart showing reflective intensity values for different tissue types.

FIG. 12 is a schematic and flow chart showing a technique for variance extraction to automatically find nerve tissue.

FIG. 13 shows the intensity variance of tissues in the time (left) and frequency (right) domains.

FIG. 14 is a flow chart of identifying tissue using an FFT technique.

FIG. 15 depicts signals used for tissue identification using phase-sensitive detection (lock-in signal detection) when: (a) phase offset=0 frame; (b) phase offset=4 frames; and (c) phase offset=6 frames.

FIG. 16 shows pictures of various rotating XPI prototypes and components. (a) 3D printed stage and gears. The polarizer and analyzer are orthogonally attached to the gears and connected to the driving gear separately so that they can remain crossed during rotation. (b) the rotating disk contains inner and outer linear polarizers that are in crossed position. (c) Early prototype of crossed-linear polarization device and light source attached to cell phone.

FIG. 17 is a chart showing a method according to another embodiment of the present disclosure.

FIG. 18. Nerve identification using FFT. (a) An original frame acquired by a rotating XPI system under the illumination of a 590 nm LED source (arrow: the chicken sciatic nerve). (b) Grayscale image of the result from FFT processing. (c) False color image of the result from FFT processing.

FIG. 19. Nerve identification using lock-in processing. (a) An original frame acquired by a rotating XPI system under the illumination of a 590 nm LED source (arrow: the chicken sciatic nerve). (b) Grayscale image of the result from lock-in processing. (c) False color image of the result from lock-in processing.

FIG. 20. Nerve identification using inter-frame calculation. (a) An original frame acquired by the rotating XPI system under the illumination of a 590 nm LED source (arrow: the chicken sciatic nerve). (b) The result of frame subtraction and averaging during the inter-frame calculation process. (c) Output as a false color map of the inter-frame calculation processing. (d) Image resulting from masking the binary mask back onto the original frames to highlight the nerve tissue.

FIG. 21. An experimental system and processing flow used for real-time experiments according to another embodiment of the present disclosure.

FIG. 22. Real-time XPI video and the processing. The frames at five different times in the original XPI video (left column) and the corresponding calculated binary nerve mask results (right column). The position of the nerve bundle is indicated with an arrow nerve in the top left frame. The solid double side arrow indicates the orientation of a polarization state generator (PSG), and the dotted line double side arow indicates the orientation of a polarization state analyzer (PSA).

FIG. 23. Frames of the real-time nerve highlighting result at five different times.

FIG. 24. XPI image of the median nerve when the PSG and the nerve bundle is at 0° (a) and 45° (b).

FIG. 25. XPI images of the cut open section of the median nerve. (a) Taken when the PSG and the nerve bundle is at 0°. (b) Taken when the PSG and the nerve bundle is at 45°.

FIG. 26. Nerve identification in a cadaver study. (a) An image of the dissected volar sided region of the arm, a small branch of the median nerve (arrow) is surrounded by fat tissues. (b) The highlighted nerve resulting from inter-frame calculation and overlaying a red mask on the nerve. (c) Grayscale image of the lock-in processing. (d) False color result of the lock-in processing.

FIG. 27. Procedure of making a fat wedge having a linearly tapered thickness from 0 to 1 mm.

FIG. 28. Left: fat wedge on top of a ruler, showing the fat layer is less transparent as the thickness increases linearly. Right: fat wedge on top of a chicken sciatic nerve fiber.

FIG. 29. Video lock-in processed result of the nerve covered by fat wedge.

FIG. 30. An illustration of fitting the curve with four data points.

FIG. 31. (a) An original frame of the chicken modal XPI video, the position of the nerve is indicated by the arrow. (b) The value map output after curve fitting with four frames.

FIG. 32. left: Image of the cadaver nerve model. Right: A value output map when different combinations of data points are chosen.

FIG. 33. A four-angle XPI system according to another embodiment of the present disclosure.

FIG. 34. Four XPI images obtained (left) and the curve fitting output (right) of a first sample chicken thigh.

FIG. 35. Four XPI images (left) and the curve fitting output (right) of a second sample chicken thigh.

### DETAILED DESCRIPTION OF THE DISCLOSURE

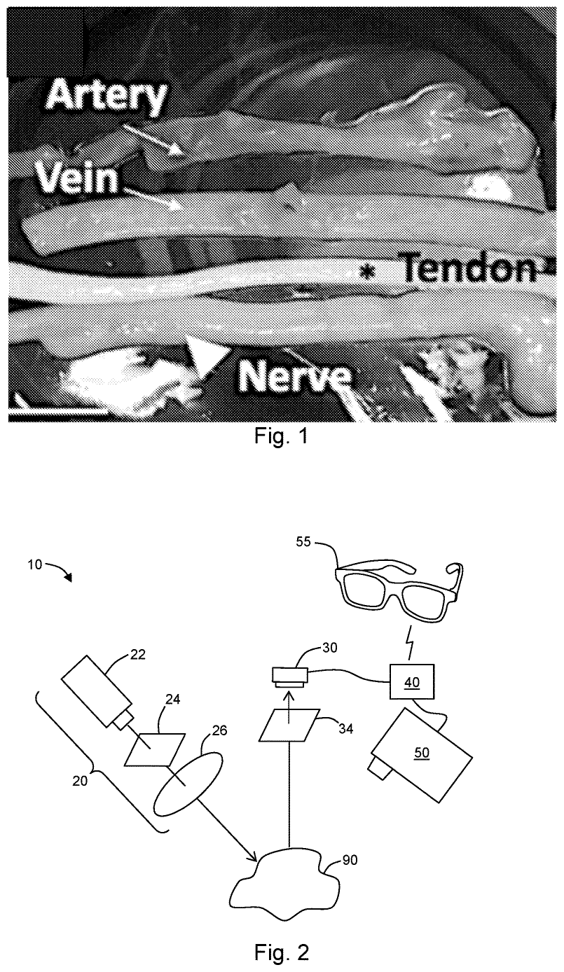

FIG. 1 is a photograph of four different tissue types and shows the difficulty in distinguishing nerve tissue from other tissue types. Polarized light has found increasing use in combination with, or as an alternative to, traditional imaging. The randomness and anisotropy of some biological tissues (e.g., plant or animal tissues) results in unique interaction with polarized light. For example, peripheral nerves are made up of a number of nerve axons wrapped by connective tissues. Such nerves have shown strong birefringence within the epineurium and perineum. The natural tension and the structure of nerve tissue can also depolarize the polarized light due to multiple scattering.

As described in recent work, the Muller Polarimetric Imaging technique can be useful in imaging and identifying nerves in animal tissue samples. The MPI approach requires three states of a polarizer and four states of an analyzer to obtain enough data so that the intrinsic retardance value of the object can be determined. However, MPI requires the use of expensive medical microscopes and polarimetric cameras, and it is difficult to develop a portable equipment for use with MPI. In other work, a surgical microscope was used demonstrate the usefulness of birefringence for nerve differentiation.

A simpler approach for revealing the polarimetric properties of biological tissues is the use of crossed polarizers. In an example, a first polarization film is used to polarize light from a light source, and a second polarization film is placed before a camera and oriented such that surface reflected light cannot pass through to the camera. In other words, such a cross-polarization system will only record the light whose state of polarization is changed when interacting the illuminated subject. A cross-polarization system can eliminate flares often seen when imaging tissue. It also has the effect of ‘highlighting’ tissues that have higher depolarization and/or birefringence, thus increasing the contrast of an image and improving image quality. Various embodiments of a cross-polarization imaging system may use linearly polarization or circular polarization. In the case of cross-linearly polarized imaging (XPI), the polarizer and the analyzer are both linearly polarized (e.g., linear polarization films), and they are placed at a non-zero angle (for example, orthogonally) to each other. In circular polarization imaging (CPI), the polarizer and analyzer may have the same handedness because the surface reflecting light will change the chirality.

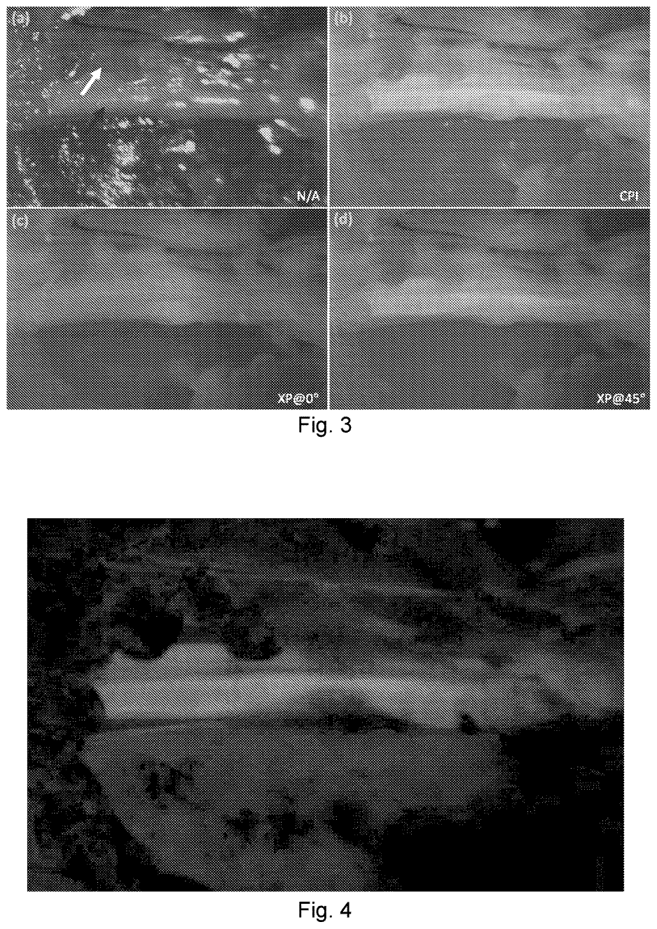

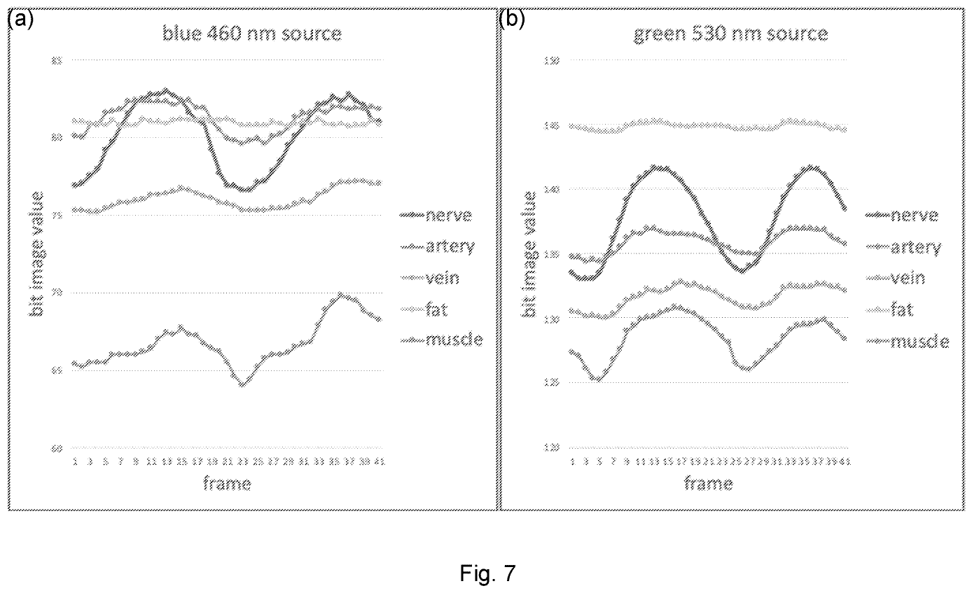

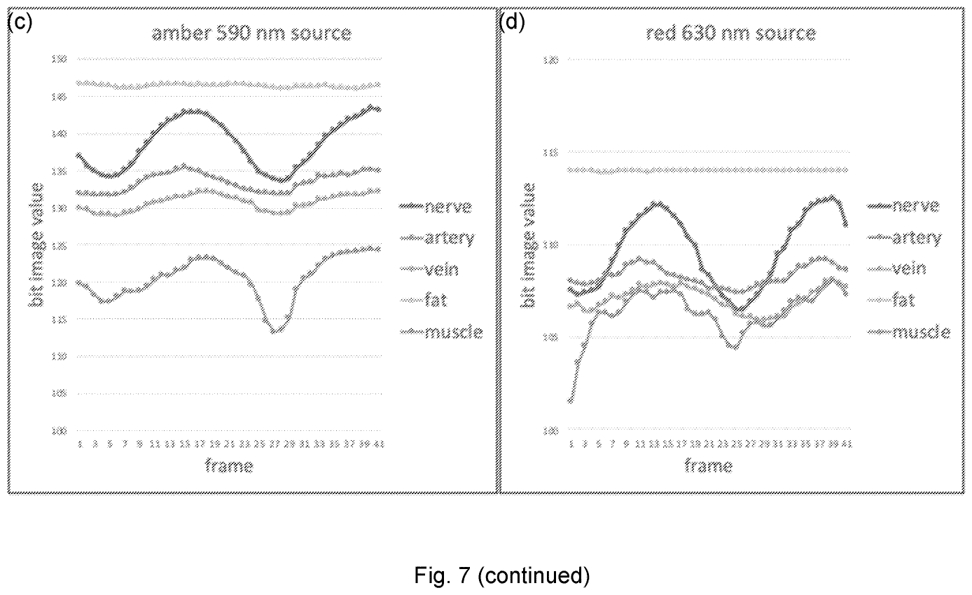

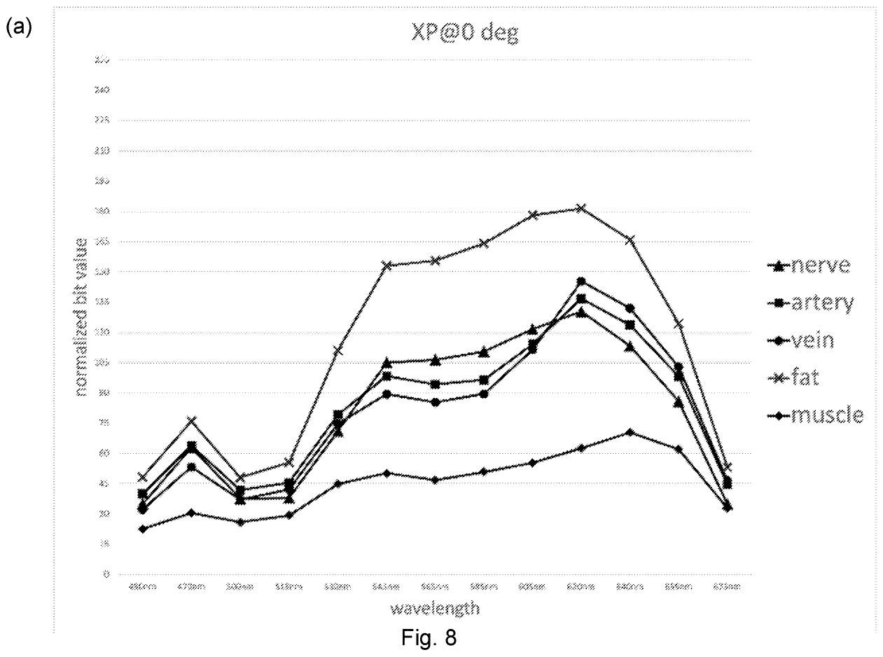

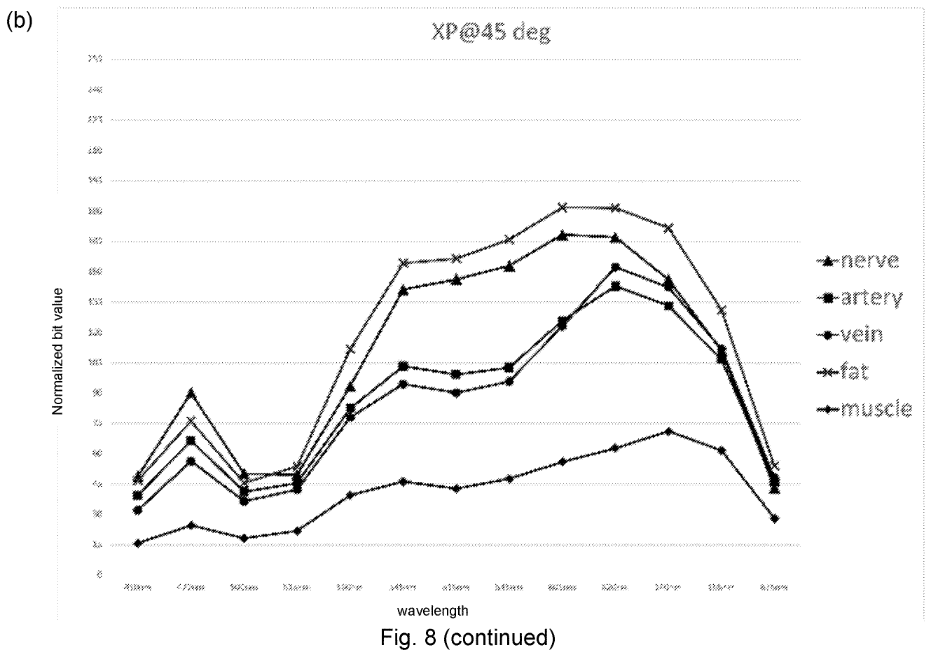

In conducting experiments using an XPI system, it was observed that polarimetric images were sensitive to the orientation of the linear polarizer with respect to certain tissues. For example, FIG. 3 shows that a nerve exhibits the strongest intensity value under XPI when the polarizer was oriented at an angle of 45 degrees to the nerve bundle (i.e., to a longitudinal axis of the nerve), which indicates the state of the linearly polarized illumination light was changed the most when the polarization direction was at 45 degrees relating to the nerve. Other tissue types, such as, for example, veins, were also observed so show some amount of dependence to the polarization angle. The intensity response of various tissues can be enhanced by selecting one or more spectral bands (e.g., colors) of the illumination light, as further described below (see also, FIG. 7—showing different variance and intensity under four different illumination light bands. In FIG. 8, the changes of intensity from 0 degree to 45 degree were obtained for thirteen spectral bands.

The present disclosure provides techniques using polarimetric imaging systems and image processing methods to automatically identify certain tissues. The presently disclosed techniques may help the surgeons quickly and accurately identify target tissues, such as, for example, peripheral nerves.

With reference to FIG. 2, the present disclosure may be embodied as a system 10 for tissue identification. The system includes a light source 20 configured to illuminate a region of interest 90 with polarized light. The region of interest 90 may be, for example, a region on, within, or partially on or within, an individual, a surgical field, etc. The light source may be inherently polarized or polarized using one or more external components such as, for example, a polarizing film, a quarter-wave plate, etc. The polarized light may be linearly polarized, circularly polarized, or elliptically polarized. In some embodiments, the polarized light is linearly polarized. For example, light from an inexpensive light source (e.g., white LED, colored LED, infrared LED, etc.) may be polarized using an inexpensive polarizer such as, for example, a polarizing film.

The light source may be a broadband light source or a source having one or more limited spectral bands. For example, the light source may include one or more spectral bandpass filters (such as a tunable filter) to limit the emitted light to one or more spectral bands having a pre-determined wavelength range. Examples of a limited spectral band may be spectral bands covering wavelength ranges (bandwidths) of <1 nm, 10 nm, 20 nm, 30, nm, 50 nm, 100 nm, 150 nm, 200 nm, or more, or spectral bandwidths between these example values. The light source may have a desired wavelength such as, for example, 460 nm (blue), 530 nm (green). 590 nm (amber), and 630 nm (red), or ranges containing a desired wavelength such as, for example, ranges centered on a desired wavelength. The light source may cover visible light, infrared, ultra violet, or portions of one or more of these. The light source may include one or more light-emitting diodes (LEDs), one or more laser diodes, lasers, halogen lights, xenon lights, metal halide lights, incandescent lights, and/or the like. The light source may include other components such as, for example, optical fibers to provide illumination to the region of interest. In the example shown in FIG. 2, the system 10 includes a light source 20 made up of a lamp 22, a polarizer 24, and a color filter (i.e., spectral bandpass filter) 26.

The light source 20 is configured to provide polarized light at more than one polarization angle with respect to the region of interest. For example, the light source may be configured to provide light at four polarization angles. In a particular example, the polarization source is configured to provide light at polarization angles of 0°, 25°, 50°, and 75° with respect to the region of interest. In some embodiments, the polarization of the light provided by the light source 20 is configured to rotate. For example, a polarization of linearly-polarized light from the light source is configured to rotate. For example, a polarizing filter may be physically rotated (e.g., using a motor, actuator, etc.) within the beam of light provided to the region of interest. In other embodiments the light source is rotated to rotate the polarization of the light. In some embodiments, the polarization rotates through at least 4 of a full rotation (i.e., at least 90°) during a sample period. In some embodiments, the polarization may rotate through at least a full rotation (i.e., at least 360°) during the sample period. The polarization may have a rotation speed of, for example, 60 revolutions per minute (RPM). 100 RPM, 200 RPM. 300 RPM, or more or less, or any speed between these example values. In some embodiments, the light source may have more than one polarizers (e.g., four polarizers) each having a different polarization from the others. In some embodiments, the light source may include more than one light source (e.g., four light sources) each having a different polarization from the others.

The system 10 includes an image sensor 30 having a field of view for acquiring images of the region of interest illuminated by the polarized light of the light source 20. For example, the image sensor may be positioned to acquire images using polarized light reflected from the region of interest. The image sensor may be a charge-coupled device (CCD), an active-pixel sensor (i.e., CMOS sensor), or otherwise. At least one analyzer 34 is positioned within the field of view of the image sensor such that light from the region of interest (for example, reflected by the region of interest) passes through the analyzer before impinging on the image sensor. The analyzer 34 has a type of polarization similar to that of the light source 20. For example, where the light source is linearly polarized, the analyzer is linearly polarized. The at least one analyzer is configured to have a polarization which maintains a non-zero angle relative to the polarization angle of the polarized light from the light source. For example, the non-zero angle may be orthogonal (i.e., 90°) with respect to the polarized light of the light source. In some embodiments, the polarization of the analyzer is configured to rotate and maintain a non-zero angle relative to the polarization of the polarized light. In some embodiments, the polarization of the analyzer is rotated to maintain orthogonality to the polarization of the polarized light from the light source. For example, the analyzer may be physically rotated in coordination with a rotation of a polarizer of the light source to maintain cross-polarization. In this way, birefringence of tissues within the region of interest may be observed (from the use of crossed polarizers) in an orientation independent manner with respect to the tissues (as a result of the continual rotation of the polarizers). In some embodiments, the at least one analyzer is made up of four analyzers, and each of the four analyzers is configured to be orthogonal to a different polarization angle of the four polarization angles of the light source.

The system 10 includes a processor 40 in electronic communication with the image sensor 30. The processor 40 is configured to obtain, from the image sensor, a series of images (frames) of the region of interest acquired using more than one polarization angle. For example, the processor may obtain 2, 3, 4, 5, 10, 15, 20, 30, 45, 60, or 100 images, or more images, or any number of images between these example values. Where the crossed polarizers are configured to rotate, higher rotational speed of the crossed polarizers may allow fast overall response (nerve identification) using the system. Along with higher rotational speed, it may be advantageous to increase acquisition rate (shorter time between each image acquisition) and/or decrease exposure time for each image. In some embodiments, the polarizer may rotate through multiple rotations and the resulting data from each rotation averaged (for example, to reduce noise in the resulting averaged data). In another example, data from a partial rotation may be averaged with one or more additional partial rotation. In embodiments having multiple fixed polarizations, the system may be configured to acquire images from using each polarization at faster sample speeds and/or with shorter intervals between samples.

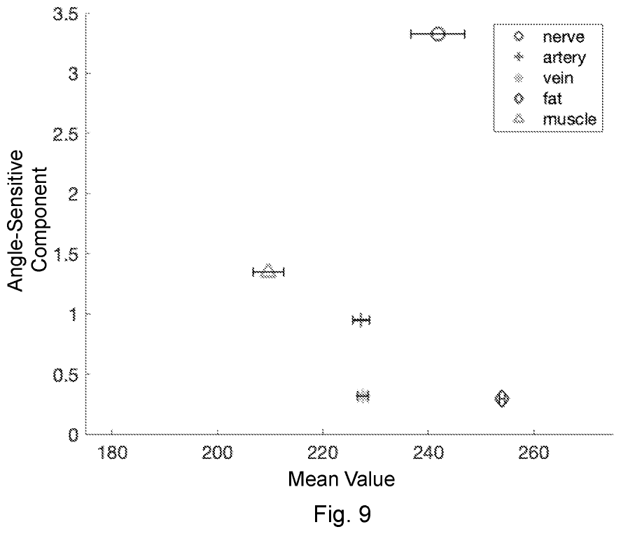

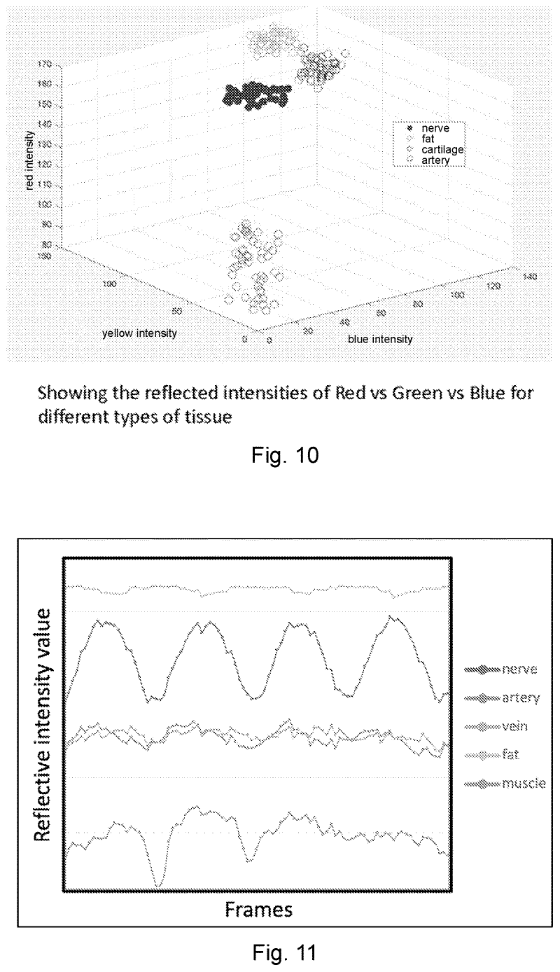

The processor identifies a tissue within the region of interest based on an intensity of one or more pixels of the series of images varying over the time series of images. For example, the different polarization angles of the polarized light may cause nerve tissue in the region of interest to vary in brightness according to each the polarization angle of the received light. As such, one or more pixels corresponding to such nerve tissue within an image of the region of interest will vary in intensity, and the processor may be configured to identify such nerve tissue based on the varying intensity of the one or more pixels. The processor may be configured to identify the tissue as one or more of nerve, artery, vein, fat, or muscle, or other tissue types. The varying intensity associated with certain tissues (e.g., nerve, vein, artery, etc.) may be distinguishable from an intensity (which may or may not vary) associated with another type of tissue (e.g., nerve, fat, artery, vein, cartilage, muscle, etc.) For example, the processor may be configured to determine a mean value of a pixel over the time series of images, and an angle-sensitive component of the pixel value (e.g., pixel value−mean value) such as in the chart depicted in FIG. 9. In this way, the pixel(s) corresponding to a particular tissue are distinguishable from pixel(s) corresponding to other tissues in the time series of images. In some embodiments, the processor may utilize a point could (for example, the three-dimensional point cloud of FIG. 10) in color space to distinguish and/or display tissue types. Additional detail regarding identifying tissue by the processor is provided below including under the heading “Image/Signal Processing.”

In some embodiments, the image sensor acquires images in one or more limited spectral bands (e.g., colors, infrared, ultraviolet, etc. and subsets of one or more of these examples). The processor may be configured to identify the tissue within the region of interest based on an intensity at the one or more spectral bands. For example, the light source may be configured to provide light in one or more limited spectral bands. The light source may be a broadband light source (e.g., white light, light source emitting light in a wavelength range of 100-1200 nm, 400-700 nm, or other desirable broadband range) and an optical filter (e.g., tunable filter, fixed color filter(s), etc.) is disposed between the light source and the image sensor (e.g., configured to be between the light source and the sample, between the sample and the image sensor, etc.) In some embodiments, color filtering may be performed by the processor. In some embodiments, the processor may obtain images from the image sensor acquired in a first limited spectral band for at least a first full rotation of the polarization of the polarized light. The processor may then obtain images from the image sensor acquired in a second limited spectral band for at least a second full rotation of the polarization of the polarized light, and (optionally) so on for additional limited spectral bands.

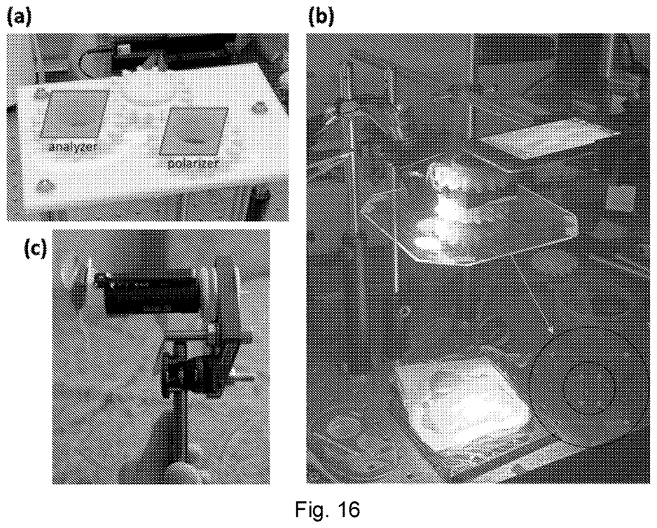

In an exemplary embodiment, a system for identifying tissue may be a portable system using a smartphone or tablet (collectively referred to herein as a smart device) (see, e.g., FIG. 16(b)). A typical smart device includes a camera (image sensor) and an LED light source. A smart-device system according to the present disclosure may use a rotating linear polarizer over the LED light source for providing polarized light to illuminate a region of interest, and a rotating analyzer over the camera. The polarizer and analyzer are configured to rotate synchronously so as to maintain cross-polarization of the light provided through the polarizer and received through the analyzer. In another example, such as that shown in FIG. 16(b), the light source and polarizer may be an external device—i.e., not a part of the smart device.

In another example, the system may use an image sensor having multiple on-chip analyzers (for example, the Sony POLARSENS™ imaging sensor). In this way, embodiments of an XPI system may eliminate some or all moving components. In some embodiments, the XPI system may be built into a wearable device such as, for example, augmented reality glasses or goggles.

In some embodiments, the processor is further configured to generate an image mask based on image pixels corresponding to the identified tissue. Such an image mask may be overlaid on images acquired by the image sensor. For example, the image mask may be structured such that an unmasked portion corresponds to the identified tissue. In some embodiments, a masked portion of the image mask corresponds to the identified tissue. The processor may be configured to alter an image or a video (e.g., a series of images, etc.) using the image mask. For example, the processor may increase an intensity of an unmasked portion of the image, add a boundary line to an unmasked portion of the image, animate an unmasked portion of the image, etc. In this way, the altered image of view may be displayed to a user, such as, for example, a surgeon, in order to assist the user in identifying tissue.

In some embodiments, the system 10 further includes a projector 50 in electronic communication with the processor 40. The projector 50 is configured to project one or more images on the region of interest 90. The processor in such embodiments is further configured to project, using the projector, a highlight on the region of interest based on the identified tissue. In the present context, a highlight can be any type of augmentation that would cause the highlighted object/area to be more easily distinguishable to a user. For example, for the projector, a highlight may be a white light, colored light, patterned light (e.g., stripes, outline of the area/object, etc.), projected arrow(s), flashing light, etc. and combinations of these or other types of augmentation. In a surgical setting, such projected highlighting would allow the surgeon to have a hands-free surgical aid.

In some embodiments, the system 10 further includes a wearable display 55 in electronic communication with the processor 40. For example, the wearable display may be a pair of augmented reality glasses or goggles, a monocle, etc. The wearable display is configured to provide augmented reality information to a wearer. The processor in such embodiments is further configured to provide, using the wearable display, a highlight overlaying the region of interest (i.e., overlaying the wearer's view of the region of interest) based on the identified tissue.

In some embodiments, the processor is further configured to identify the tissue as abnormal tissue. For example, it has been found that physically damaged tissue or diseased tissue may have polarization-related properties different from those of normal tissue. For example, stretched nerves, pinched nerves, diseased nerves (e.g., diabetic nerves), etc. may behave differently under polarized light from normal nerve tissue. As such, the present system may be used to differentiate abnormal tissue from normal tissue. In this way, the present system may be used as a diagnostic aid.

The processor 40 may be in communication with and/or include a memory. The memory can be, for example, a random-access memory (RAM) (e.g., a dynamic RAM, a static RAM), a flash memory, a removable memory, and/or so forth. In some instances, instructions associated with performing the operations described herein (e.g., identifying a tissue, etc.) can be stored within the memory and/or a storage medium (which, in some embodiments, includes a database in which the instructions are stored) and the instructions are executed at the processor.

In some instances, the processor includes one or more modules and/or components. Each module/component executed by the processor can be any combination of hardware-based module/component (e.g., a field-programmable gate array (FPGA), an application specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general purpose processor, a central processing unit (CPU), an accelerated processing unit (APU), a field-programmable gate array (FPGA), an application specific integrated circuit (ASIC), a digital signal processor (DSP), and/or the like.

It should be noted that the presently-disclosed systems and methods may be used for exposed tissues and/or tissues below the surface of the region of interest. For example, the birefringent properties of nerves are visible even where the nerve is beneath other tissue (e.g., beneath fat tissue).

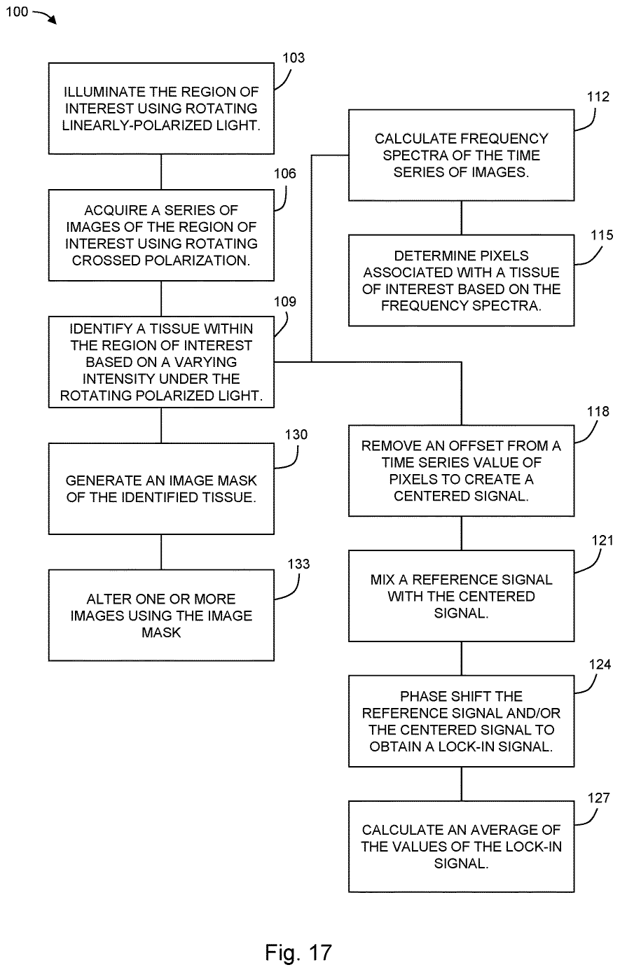

With reference to FIG. 17, in another aspect, the present disclosure may be embodied as a method 100 for tissue identification. The method 100 includes illuminating 103 a region of interest with a linearly-polarized light configured to have at least four polarization angles relative to the region of interest. The region of interest may be, for example, a region on, within, or partially on or within, an individual, a surgical field, etc. The illumination may be provided using a light source that is inherently polarized or polarized using one or more external components such as, for example, a polarizing film, a quarter-wave plate, etc. For example, light from an inexpensive light source (e.g., white LED, colored LED, infrared LED, etc.) may be polarized using an inexpensive polarizer such as, for example, a polarizing film.

The linearly-polarized light may be a broadband light or may be a light having one or more limited spectral bands. Examples of a limited spectral band may be spectral bands covering wavelength ranges (bandwidths) of <1 nm, 10 nm, 20 nm, 30, nm, 50 nm, 100 nm, 150 nm. 200 nm, or more, or spectral bandwidths between these example values. The light may have a desired wavelength such as, for example, 460 nm (blue), 530 nm (green), 590 nm (amber), and 630 nm (red), or ranges containing a desired wavelength such as, for example, ranges centered on a desired wavelength. The light may comprise or consist of visible light, infrared, ultra violet, or portions of one or more of these.

A series of images of the region of interest is acquired 106 using an image sensor. The images are acquired through an analyzer having a polarization configured to have at least four polarization angles which are non-zero (e.g., orthogonal) to the polarization angle of the linearly-polarized light. For example, the polarization of the analyzer may rotate synchronously with the linearly-polarized light such that the light received by the image sensor is cross polarized. In some embodiments, the polarization rotates through at least ¼ of a full rotation (i.e., at least 90°) during a sample period. In some embodiments, the polarization may rotate through at least a full rotation (i.e., at least 360°) during the sample period. The polarization may have a rotation speed of, for example, 60 revolutions per minute (RPM), 100 RPM, 200 RPM. 300 RPM, or more or less, or any speed between these example values. The series of images may be obtained in one or more limited spectral bands. For example, the images may be acquired in a first limited spectral band for at least a first full rotation of the polarization of the polarized light, and acquired in a second limited spectral band for at least a second full rotation of the polarization of the polarized light.

The method includes identifying 109 a tissue within the region of interest based on an intensity of one or more pixels of the series of images varying according to the polarization angle of the rotating linearly-polarized light. In some embodiments, the identified 109 tissue is one or more of nerve, artery, vein, fat, and muscle tissue. In some embodiments, the identified 109 tissue is nerve tissue. The method may include distinguishing tissue types in the region of interest based on differences in the variability of intensity associated with tissue types. In embodiments where at least some of the images are acquired in one or more limited spectral bands, the issue may be identified based on an intensity when illuminated by the one or more limited spectral bands. The tissue may be identified as abnormal tissue (e.g., diseased or physically damaged) based on the varying intensity of the tissue. In some embodiments, an orientation of the identified tissue may be determined based on an intensity of the tissue corresponding to a rotational position of the rotating linearly-polarized light, and relative to the varying intensity of the tissue.

In some embodiments, the tissue may be identified by calculating 112 frequency spectra of the time series of images obtained from the image sensor; and determining 115 pixels corresponding to the tissue based on a peak amplitude of a frequency corresponding to the tissue. In some embodiments, the tissue is identified by removing 118 an offset from a set of time series values of a pixel to generate a centered signal for the pixel; mixing 121 a reference signal with the centered signal for the pixel: shifting 124 the phase of the reference signal and/or the centered signal to determine a phase-locked signal; and calculating 127 an average value of the phase-locked signal.

The method 100 may further include generating 130 an image mask based on image pixels corresponding to the identified tissue. An image or a series of images may be altered 133 using the generated 130 image mask. Altering 133 the image or series of images includes one or more of increasing an intensity of an unmasked portion of the image, drawing a boundary line around an unmasked portion of the image, changing a color of an unmasked portion of the image, and animating an unmasked portion of the image.

In some embodiments, a highlight may be projected on the region of interest based on the identified tissue. In some embodiments, augmented reality information may be provided to a wearable display. For example, the augmented reality information may be a highlight overlaying the region of interest based on the identified tissue.

In another aspect, the present disclosure may be embodied as a non-transitory computer-readable medium having stored thereon a program for instructing a processor to: illuminate a region of interest with a rotating linearly-polarized light; obtain a series of images of the region of interest using an image sensor via an analyzer rotating synchronously with the linearly-polarized light such that the light received by the image sensor is cross polarized; and identify a tissue within the region of interest based on an intensity of one or more pixels of the series of images varying according to the polarization angle of the rotating linearly-polarized light.

In some embodiments, the method include detecting a surgical instrument within the field of view (e.g., near the region of interest). The method may include generating an alert when the surgical instrument approaches the identified tissue (e.g., nerve tissue). For example, when a surgical instrument becomes closer to the identified tissue than a pre-determine distance, an audible alarm may be sounded, a visible indicator may be displayed, and/or haptic feedback may be provided to an operator (e.g., a surgeon or assistant). In an example, an indicator may be displayed within augmented reality goggle. In another example, an ambient light or a light illuminating the surgical field may change color to indicate proximity to the identified tissue. These are non-limiting examples, and other techniques for alerting an operator may be used.

Image processing may be performed within a device according to the above disclosure (e.g., a wearable device) or may be performed separate from the device. For example, the processor may be within an operating room where the XPI system is being used, and the image processing may be performed using this in-room processor. In another example, the processor may be located within a same building or campus, or may be a cloud processor. The processor may communicate with the image sensor via wired and/or wireless communication.

### Image/Signal Processing

To make use of the angle dependence of the nerve in XPI system, several experimental embodiments were tested using several real time image/video processing methods. The below are non-limiting example techniques. Other techniques may be used and are within the scope of the present disclosure.

### Inter-Frame Subtraction and Averaging (Inter-Frame Calculation)

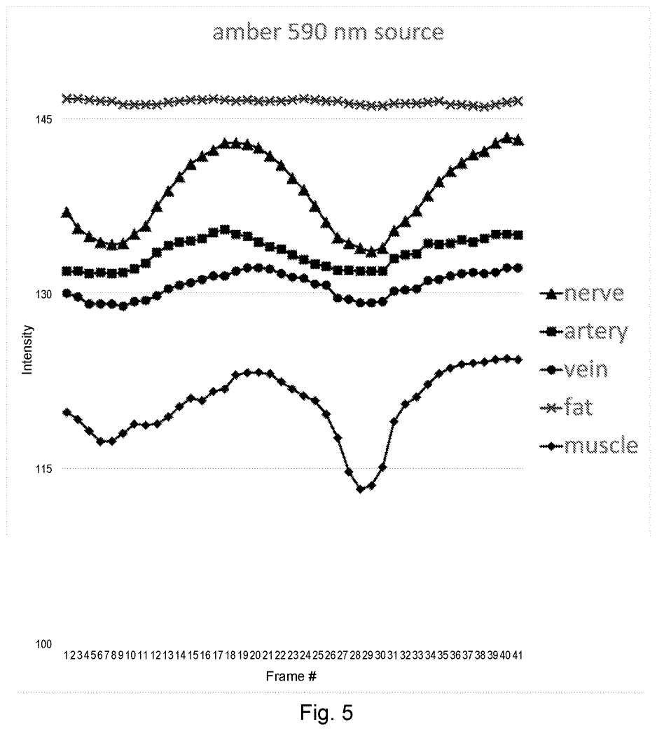

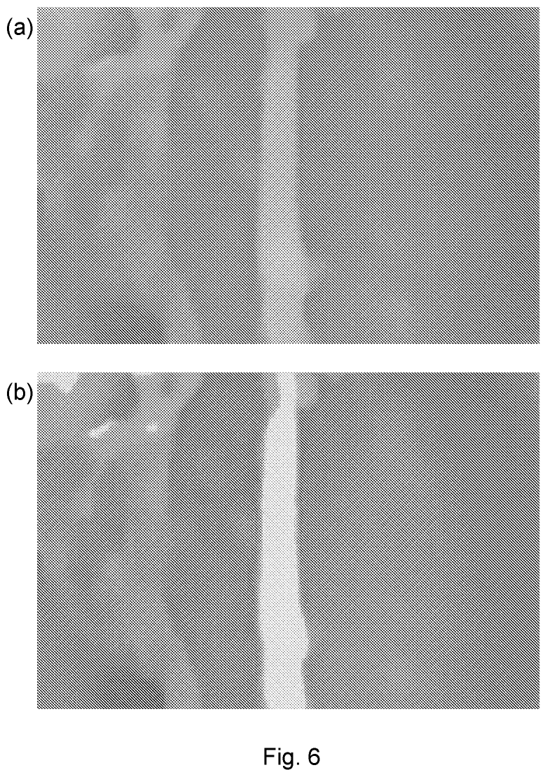

In an example, an image subtraction was performed between the most highlighted nerve images (XPI@45 deg-XPI@0 deg)(see FIG. 3), which obtained the shape of the nerve, as shown in FIG. 4. Then, the periodically varying intensity plots for the tissues was obtained using a system as described herein. FIG. 5 shows that the intensity of the nerve varied periodically with the rotation of the XPI system. FIG. 6(a) shows an example frame of the experimental time series images. The recorded video (time series of images) was processed by frame, so that the highlighted nerve signal could be extracted by calculation of the frames in each rotating period. This was then applied to the original video in another color (for example, green in the case of the experiment performed, which is shown as increased brightness in FIG. 6(b)). This applied highlighting serves as a visualization aid to remind the operator.

In some embodiments, the processor identifies a tissue using inter-frame subtraction and averaging. For example, with reference to FIG. 11, from the intensity plots of different tissues in a region of interest illuminated by amber light, it can be seen that although all the tissues have some amount of variance in reflective intensity relating to polarization angle, the nerve tissue has the greatest variance and is the most sinusoidal. Inter-frame processing can be applied to extract the variance, followed by averaging to obtain a stabilized signal. A threshold is then applied, where the threshold is configured such that only nerve tissue signal remains. In this way, nerve tissue may be identified by the processor.

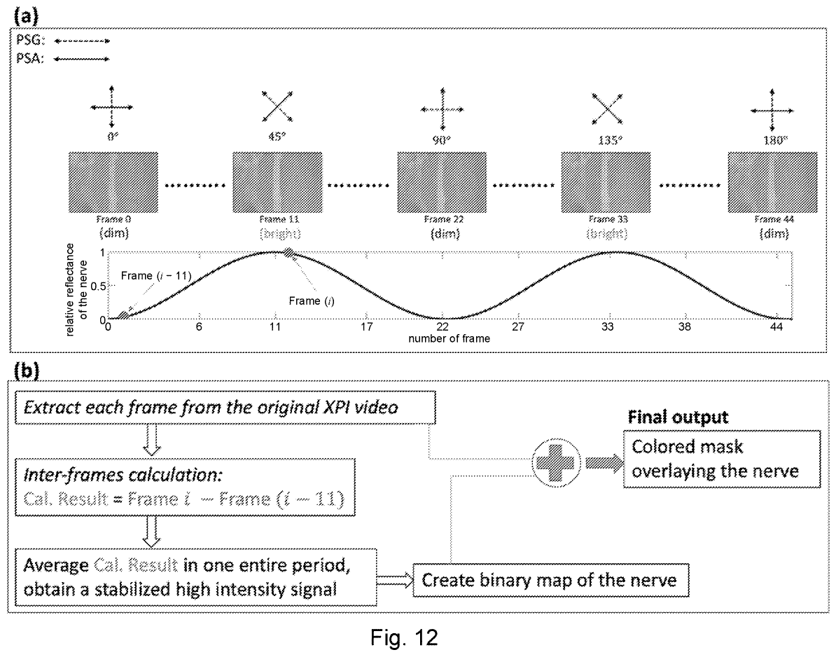

To illustrate the concepts of a method according to the present disclosure, in FIG. 12(a), the variance of the nerve intensity is approximated to a sin² function, whose distance between the trough and the nearest peak is about 11 frames. Based on this ‘frame distance,’ and the goal of extracting the difference in intensities, all the frames of the XPI time series images are processed according to the following (which is depicted in FIG. 12(b)). First, interframe subtraction is performed on the time series images—Frame(1)-Frame(i−11), from i=12 to the last frame. This provides calculation results (termed “Cal. Result” in the figure) as (Nframes−11), where Nframes is the total number of frames (images) in the time series. Because the subtraction is not always done between the peak and the trough, the calculation results may be averaged over one full rotation of the crossed polarizers to produce a set of frames that would form a more stable video showing the degree of variance in the intensities. Finally, a color map can be built to show how much the intensities of the tissues vary with the rotation of the polarization. In a particular embodiment, the nerve tissue has the highest value, and may be identified by using a threshold. In this way a binary mask can be made and/or the data corresponding to the identified tissue may be exploited.

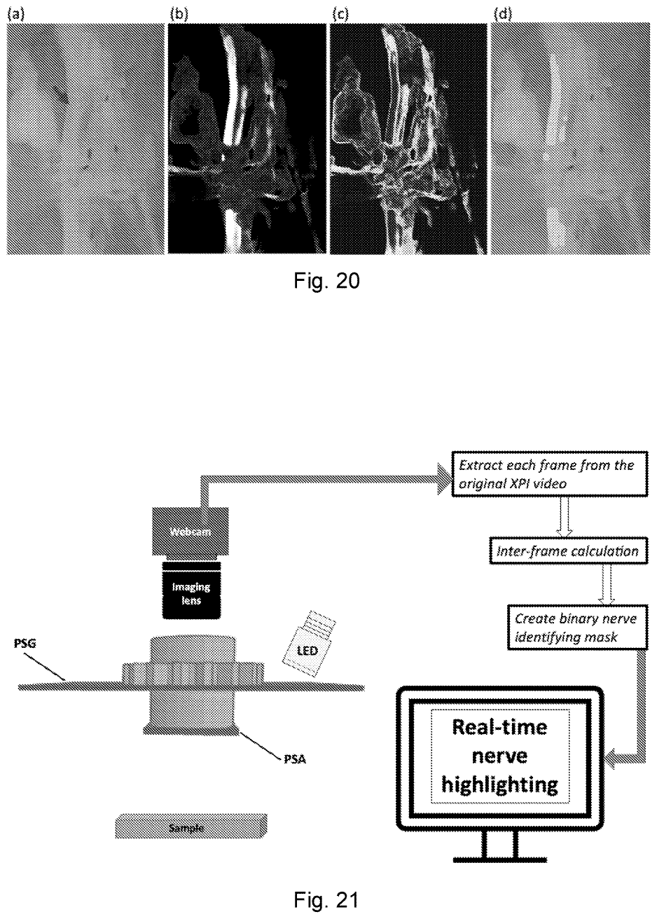

FIG. 20 shows the results of nerve tissue identification using an inter-frame calculation as described in this section. The inter-frame calculation was performed using 88 frames extracted from a rotating XPI video of a sample. FIGS. 20(b) and 20(c) are the normalized gray scaled and false color image of the calculation result—the high intensity area indicates the existence of exposed nerve tissue, while low intensity portions are other tissues. To serve as a visual reminder to the operator, a binary mask can also be generated by thresholding the frame calculation results and then applied back to the original image resulting in the image shown in FIG. 20(d).

### Pixel Value Fast Fourier Transform (FFT)

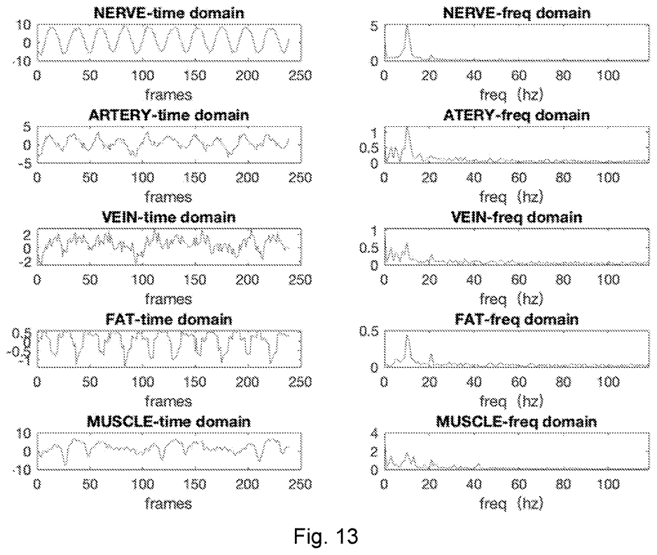

The intensity plots of some tissues will vary periodically over the time series of images obtained by the processor (periodic intensities in the time domain). FFT was applied to the intensity profiles of different tissue types and differences in the frequency domain were observed. In an experimental embodiment, time domain plots of five types of tissue were obtained and are shown on the left side of FIG. 13. FFTs of these time domain data were calculated and are shown on the right side of FIG. 13. Each of the frequency domain plots in the experimental embodiment had a peak at a common frequency (related to the rotational speed of the crossed polarizers—e.g., four peaks per 360° rotation). The vein, fat, and muscle tissue show other peaks in the frequency spectrum as well. It was noted that the nerve tissue shows the strongest peak at the common frequency as compared to the others. This allows the use of a threshold in order to differentiate nerve tissue from other tissues.

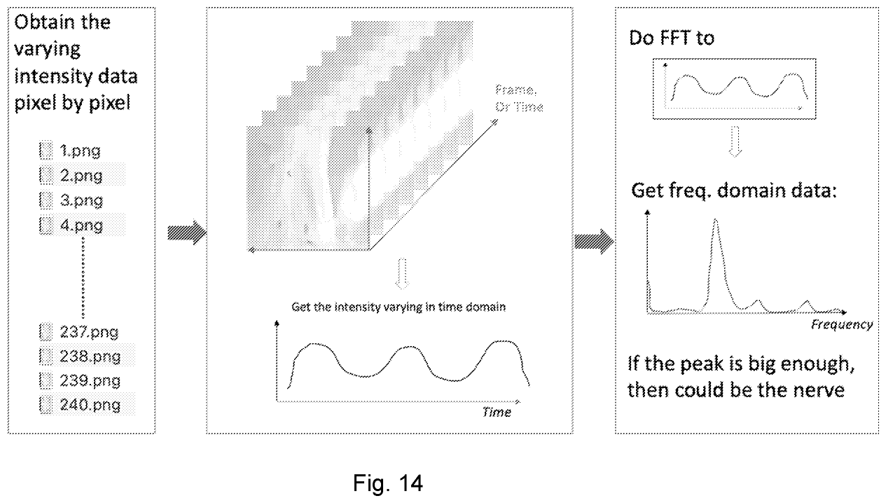

In some embodiments, the processor is programmed to identify a tissue using an FFT technique. In other words, the processor identifies a tissue by determining a frequency spectra of some or all of pixels of the time series of images. An exemplary embodiment is shown in FIG. 14. A relatively large number of frames was used to generate a time domain plot for each selected pixel. An FFT is performed for each of the selected pixels to determine the frequency spectrum for each. It should be noted that the data need not be plotted (visualized, graphed, etc. in either the time domain or frequency domain. In some embodiments, an amplitude is determined at the common frequency peak. A threshold amplitude is set to determine which pixels correspond to a tissue of interest (e.g., a nerve tissue). The FFT approach reveals a true frequency domain property of the tissues under a rotating XPI system, and this approach is less dependent on the light source used.

The pixels determined to correspond with the desired tissue can be used to, for example, create an image mask, such as a binary mask. It should be noted that, while the approach has been described as acting pixel-by-pixel, it may be performed on a subset of pixels. For example, the approach may be performed on every second pixel, every third pixel, every fourth pixel, every fifth pixel, etc. or other subsets, patterned or not patterned. In some embodiments, once the subset of pixels has been processed, the processor may be configured to analyze additional pixels. For example, the above pattern may be filled in to analyze additional pixels according to the pattern. In another example, additional pixels are analyzed between pixels which have been determined to be different from each other. In this way, the edges of each tissue can be determined.

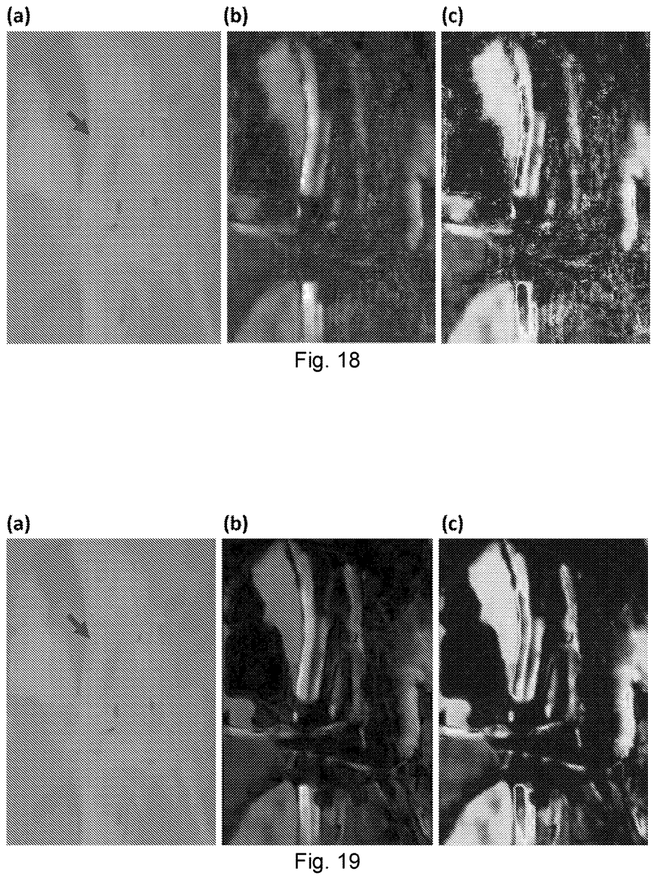

The results of an additional FFT experiment are shown in FIG. 18. The output peak values of the frequency domain signal for every pixel are displayed in the grayscale map shown in FIG. 18(b). Although FFT processing is a relatively time-consuming process, it can be seen that the pixels located at the area of the nerve tissue have higher values, which can be seen more clearly in the false color map of FIG. 18(c).

### Lock-In Signal Detection

In another embodiment, phase-sensitive detection (using lock-in amplification) may be used to resolve a tissue signal in the obtained series of images. This approach can be used to select the signal of varying intensity reflected from the desired tissue—e.g., nerve tissue.

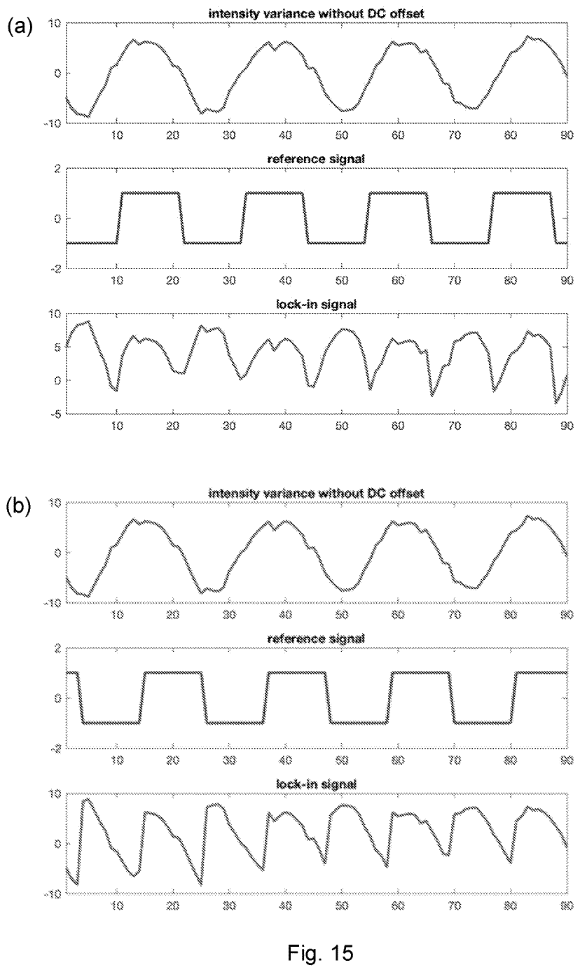

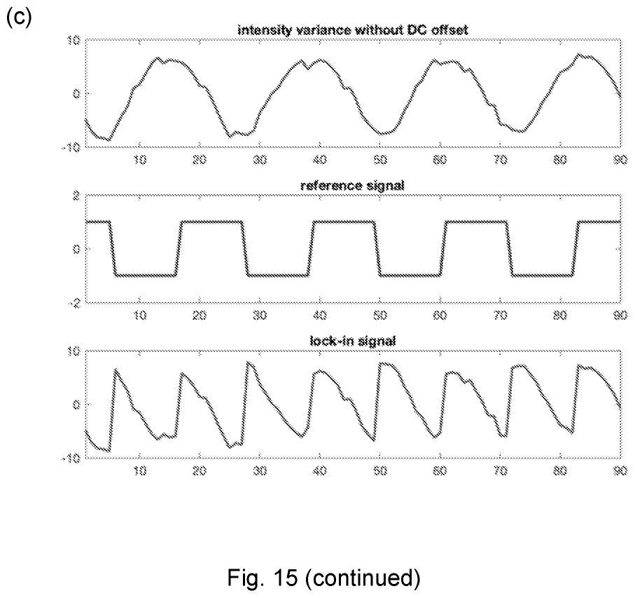

For each selected pixel, a mean value of the time domain series of the pixel is calculated and then subtracted from each value of the pixel. In this way, the time series data of the pixel are re-centered around a 0 value (e.g., an offset is removed). A reference signal is generated having a frequency based on the known rotational speed of the polarization. The reference signal may alternate between values of −1 and 1 (as shown in FIGS. 15A-15C). Other reference signals suitable for use with lock-in amplification can be used. The reference signal is mixed (i.e., multiplied) with the time series pixel data, and a phase of the reference signal is varied relative to the frames of the image data (e.g., the phase of the reference signal may be adjusted, the time series data may be shifted, or both) to lock-in the desired signal. The signal is locked-in at the phase shift which maximizes the average value of the mixed time series data. A tissue of interest can then be selected based on the average value of the locked-in signal. For example, because nerve tissue has been found to exhibit the largest variance due its polarization-related properties (e.g., birefringence, etc.), nerve tissue will show the highest average values for its locked-in signal (see, for example, Table 1).

TABLE 1
Average value of lock-in signals for varying tissue
and (for nerve tissue) varying phase offset.

| | | |
|---|---|---|
| | Phase | Average value of |
| Tissue | offset | the lock-in signal |

| | | | |
|---|---|---|---|
| Nerve | 0 | frame | 4.2089 |
| Nerve | 4 | frames | 2.3222 |
| Nerve | 6 | frames | 0.0933 |
| Artery | 0 | frame | 1.1853 |
| Vein | 0 | frame | 0.3744 |
| Fat | 0 | frame | 0.3315 |
| Muscle | 0 | frame | 1.5675 |

Additionally, because the final output of the lock-in processing is dependent on the phase offset between the reference signal and the original signal, the orientation of some tissues may be determined. For example, knowing the peak reflective intensity of nerve tissue appears at when the polarization is at 45 degrees to the longitudinal axis of the nerve, the orientation of the nerve can be determined based on the reference signal phase offset.

An additional experiment using lock-in processing was performed and the results are shown in FIG. 19. A frame from the original XPI video is displayed in FIG. 19(a), showing only the reflective intensity at the point that the frame was taken. By performing lock-in processing on the signal of each pixel, an output two-dimensional map was generated showing the processed AC values of each pixel. FIG. 19(b) shows that the values of the pixels correlated to the nerve were high and appear white in the grayscale image, and a colored AC value map was generated (FIG. 19(c)) indicating the existence of the nerve tissue in the high intensity areas.

### Real-Time Nerve Finding

It is shown above that all the three methods can successfully identify nerve tissue in the final outputs. In some embodiments, inter-frame calculation can be advantageous for real-time practice since it skips the step of transferring the video frames into arrays of pixel values, and the nerve can be recognized by direct frame calculating and thresholding out the periodic varying signals.

FIG. 21 shows a rotating XPI system used for preliminary real-time testing. The example system used for the real-time experiments included a grayscale webcam (recording at 30 fps and display in real-time) connected to a laptop computer and a motor operating at 20 rpm. The processing technique used for the experiments is shown in the flow chart at the right side of FIG. 21.

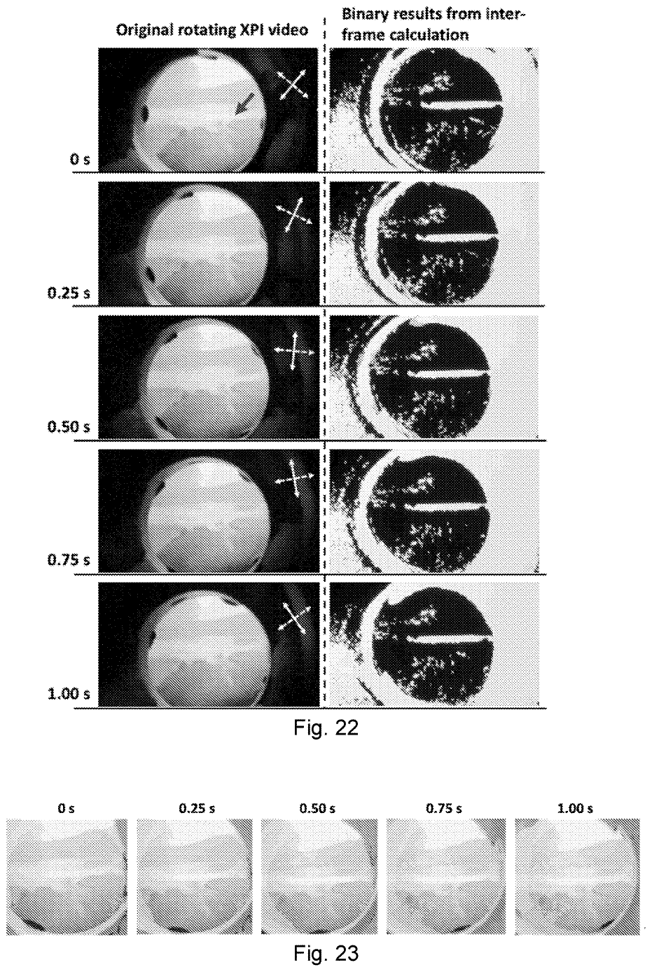

The frames of the original XPI video and the corresponding inter-frame calculation results are shown in FIG. 22, which shows frames at five different times. As the times move from 0 s to 1.00 s, the angle between the nerve bundle and the polarization state generator (PSG) varies at a constant speed, and the nerve (arrow in top frame) in the original XPI video exhibits a variation of its intensity. In the frames at time 0 s, the nerve is the most highlighted since the PSG is at approximately 45 degrees with the nerve, while in the frames at time 0.75 s, the nerve bundle appears dimmer when the angle between the PSG and the nerve is approximately 90 degrees. However, the calculated binary results shown in the frames of the right side of the figure can always maintain the shape of the nerve bundle. The binary results can be applied back to the original frames in real-time.

FIG. 23 shows the final output video at the same five times. The position of the nerve is highlighted throughout these frames by the calculated binary mask. During the experiment, the resulting video was able to be displayed on a monitor with a delay of approximately 0.5 s from the acquisition of the original rotating XPI video.

### Experimental Embodiment—Cadaver Study

This research project adheres to strict ethical guidelines to ensure appropriate consent and reverence for deceased individuals and their families when using cadavers. The ultimate goal of this study is to advance medical knowledge and enhance human health while acknowledging the immeasurable contribution made by the donors. Our unwavering commitment to maintaining the utmost levels of openness, dignity, and sensitivity throughout the research process reflects our appreciation for the donors' selfless acts. Donors are procured in compliance with the Uniform Anatomical Gift Act (UAGA) and the National Organ Transplantation Act (NOTA), and informed consent is obtained from either the donor themselves or their legal next-of-kin in all instances. All tissues are fresh-frozen without the use of any preservatives. In accordance with HIPAA regulations, no identifying information about the donors will be disclosed under any circumstances.

### XPI Result of the Median Nerve

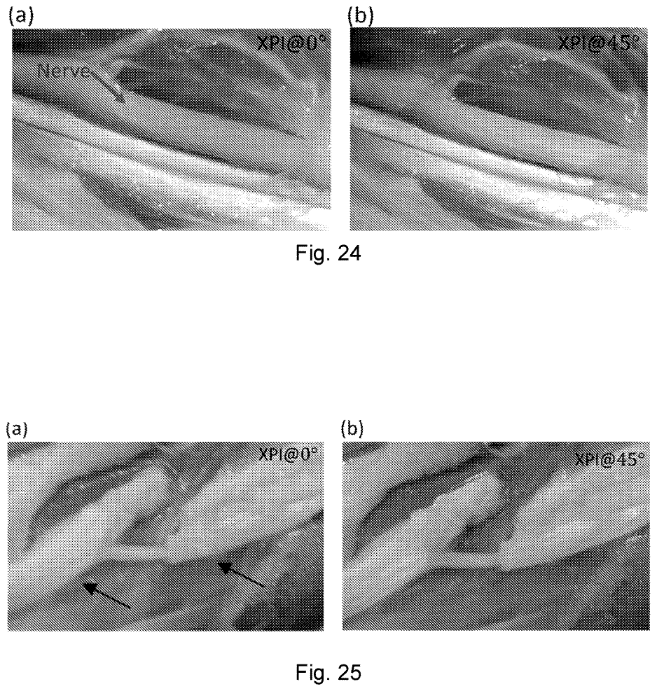

To study the reflective intensity of the human peripheral nerve using embodiments of the present XPI system, a cadaver arm was first dissected to show the main branch of the median nerve, and XPI images of the median nerve sample were acquired both with the PSG is at 0 degrees and 45 degrees to the nerve bundle while keeping the PSA in an orthogonal relationship with the PSG. The images obtained are shown in FIG. 24. It can be seen that the surface of the median nerve is highlighted (shows higher intensity) when the angle between the nerve bundle and the PSG is changed from 0° to 45°.

To show the polarization angle sensitivity was caused by the epineurium of the nerve, the median nerve sample was cut open and another pair of XPI images are obtained at 0° and 45°. FIG. 25 shows that when changing the angle between the nerve bundle and the PSG from 0° to 45°, only the outer layer of the nerve bundle was highlighted, where the arrows indicate the highlighted epineurium of the nerve.

### Nerve Recognition Results in Arm Study

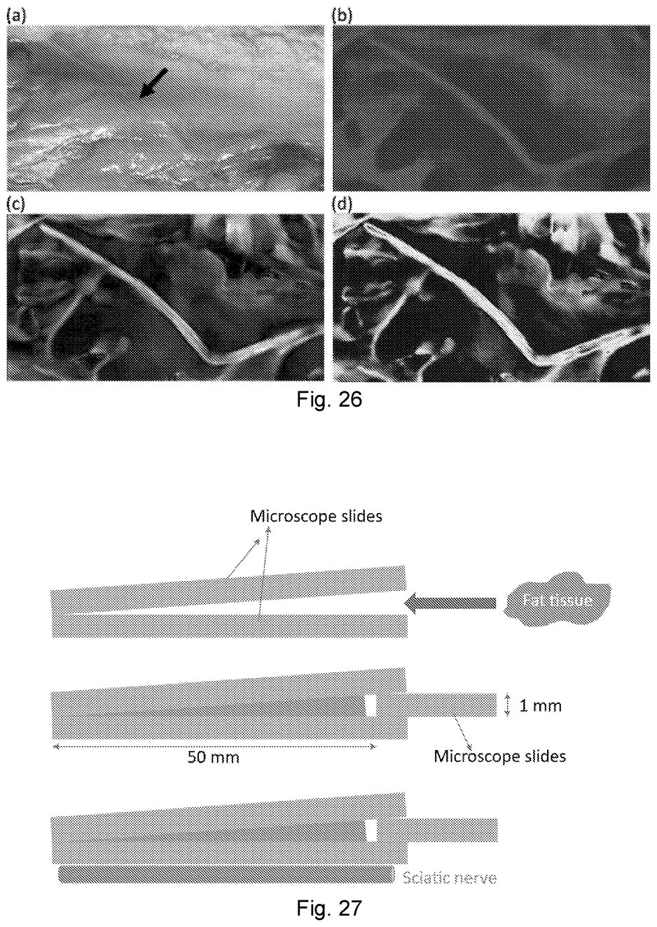

A cadaver arm study was also conducted to show the feasibility of the processing methods described above. Firstly, a dissection on the volar side of the upper left arm was performed by a surgeon, exposing the region of interest, a small branch of the median nerve surrounded by other tissues and covered by a thin fat later (FIG. 26(a)). Due to the similar colors of the nerve and the surrounding tissues, the nerve bundle cannot be seen clearly, which might lead to accidental injury of the nerve if further dissection is performed. A multispectral XPI System with a 460 nm source to acquire videos for further processing.

Frames from the rotating XPI video were processed using the inter-frame calculation technique described above. FIG. 26(b) shows a frame with the highlighted nerve obtained using frame calculation and applying a resulting red mask to the original nerve image. The nerve bundle was mostly covered, and the nearby fat tissues were relatively dark in the 460 nm illumination. A grayscale image and false color image of result from the video lock-in processing method are shown in FIGS. 26(c) and 26(d), respectively, from which it can be seen that the nerve tissue stands out clearly from the background tissues. In this instance, the nerve was located several hundred microns below other tissue. The ability to differentiate the nerve at different depths was studied using linearly tapered fat wedge.

### Experimental Embodiment—Identifying Nerve Under Fat Layer.

In addition to experiments conducted on exposed nerve tissues, the depth dependence of differentiation was studied using a fat tissue layer which was linearly tapered in thickness. Using an embodiment of the present rotating XPI imaging system, a fat wedge was placed on top of the nerve fiber to simulate the instance where the nerve is buried in fat tissues of different thicknesses.

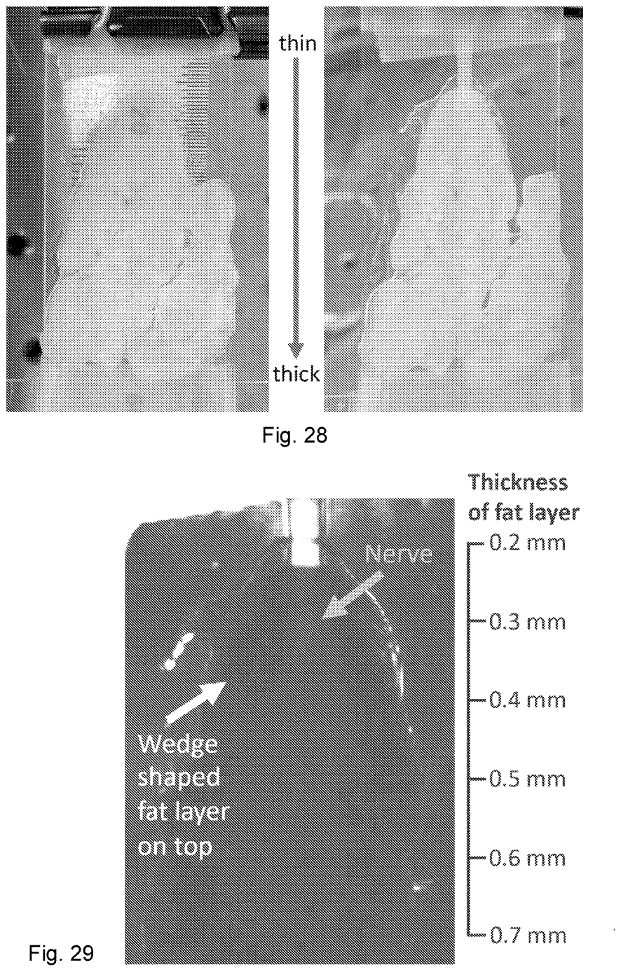

The procedure used to make a linearly tapered fat wedge is shown diagrammatically in FIG. 27. Two 1 mm thick microscope slides were used to create a wedge-shaped space where the fat tissue was placed, and another 1 mm thick slide was used to constrain the thickest side of the wedge. The fat tissue was then pressed to fill the wedge-shaped space and thus a fat layer with linearly tapered thickness (0~1 mm) was formed. The nerve was then placed under the fat wedge (FIG. 28) and imaged with the rotating XPI system.

After recording the rotating XPI video of the fat wedge covered nerve, lock-in processing of the video was performed. A frame from the resulting video is shown in FIG. 29. FIG. 29 shows that the entire fat layer possesses a low intensity after the processing, while the exposed part of the nerve fiber is highlighted in the output video as was expected. For the buried parts of the nerve, differentiation of the nerve was achieved at up to a 0.4 mm~0.5 mm thick fat layer. The results from the varying fat layer imaging shows the potentials of subsurface imaging of the presently-disclosed technique.

### Further Discussion—Curve Fitting of the Varying Intensity

In the above experimental embodiments, nerve identification was performed using the three types of video frame processing methods and based on a large amount of data. In other words, the varying intensities were directly extracted from the high speed (240 fps) recorded video, which can bring heavy loads to the data saving and processing procedures. In some embodiments, it may be advantageous to limit the processing and storage requirements.

With the knowledge that the object to be identified, the nerve tissue, shows a sine wave-like intensity profile under rotating XPI, the varying intensity profiles of the nerve tissue can be fitted out from a reduced number of data points. In this case, the sine wave can be described in the equation:

$$y = a * sin ( \frac{x}{f} - b ) + c \qquad ( 1 )$$

where x is the number of frames, y is the value of intensity, and f is a known frequency value which shows the speed of the rotation of the XPI system. There are three unknown coefficients left: coefficient b shows the phase of the intensity curve, coefficient c shows the average intensity value, and coefficient a indicates how the periodic variation goes.

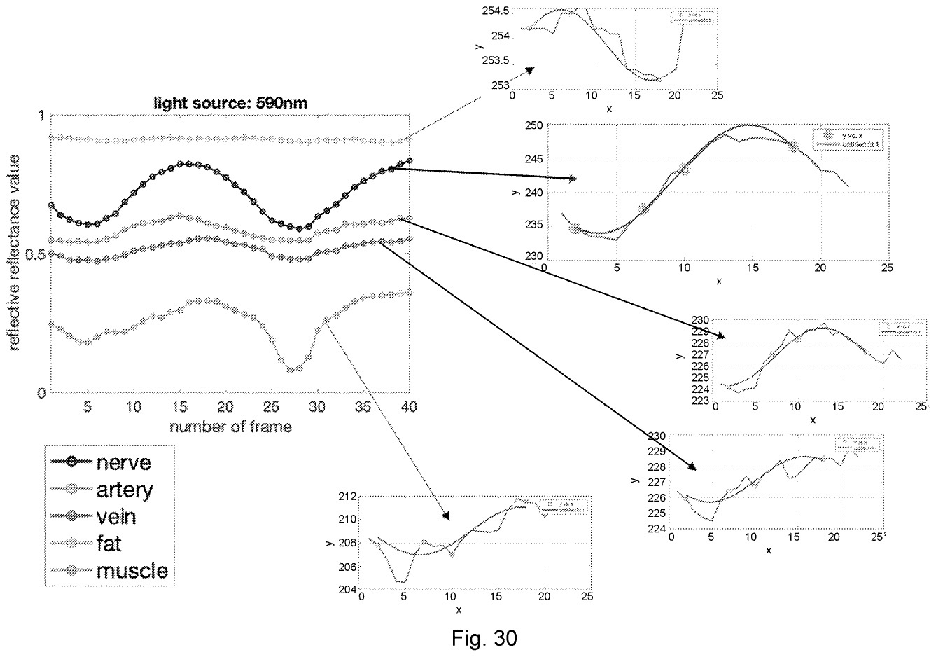

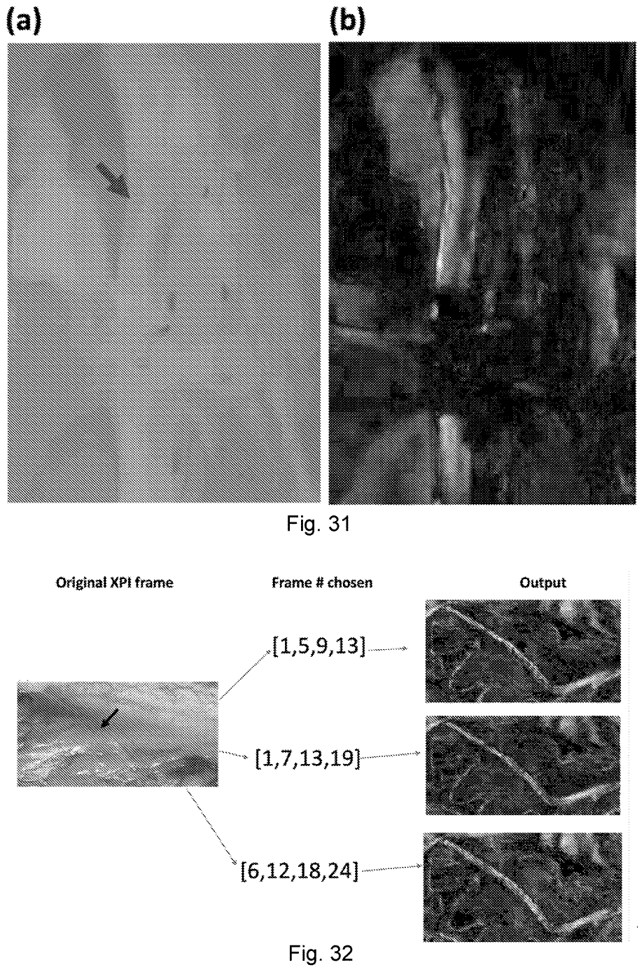

To derive the three unknown coefficients and successfully fit the curve, theoretically, four well-chosen data points will be more than enough, meaning, four pairs of x and y values can let us fit the curve. As shown in FIG. 30, four chosen data points (shaded circles) be used to fit a sine curve.

To further study the use of reduced set of data points, the combinations of data points were set in different equidistance and different phase. The combinations of data points (frames of the XPI video) and the corresponding coefficient a values for different types of tissue are shown in Table 2 (equidistance) and Table 3 (phase).

TABLE 2
the combinations of data points chosen in different equidistance
and the output coefficient a value for different types of tissue.

| Chosen Frame | Nerve | Artery | Vein | Fat | Muscle |
|---|---|---|---|---|---|
| 1) 1, 2, 3, 4 | 25.21 | 24.67 | 25.67 | 24.61 | 24.77 |
| 2) 1, 3, 5, 7 | 24.17 | 28.54 | 23.93 | 24.53 | 24.22 |
| 3) 1, 4, 7, 10 | 25.81 | 23.43 | 1.615 | 25.51 | 23.24 |
| 4) 1, 5, 9, 13 | 8.157 | 3.127 | 2.029 | 0.2142 | 2.701 |
| 5) 1, 6, 11, 16 | 7.113 | 2.412 | 1.15 | 0.6247 | 1.976 |
| 6) 1, 7, 13, 19 | 7.477 | 2.626 | 1.533 | 0.5864 | 1.61 |
| 7) 1, 8, 15, 22 | 6.884 | 2.034 | 0.752 | 0.7018 | 1.251 |

TABLE 3
the combinations of data points chosen in different phase and
the output coefficient a value for different types of tissue.

| Chosen Frame | Nerve | Artery | Vein | Fat | Muscle |
|---|---|---|---|---|---|
| 1) 1, 6, 11, 16 | 7.113 | 2.412 | 1.15 | 0.6247 | 1.976 |
| 2) 2, 7, 12, 17 | 7.627 | 2.559 | 1.368 | 0.6172 | 1.996 |
| 3) 3, 8, 13, 18 | 7.996 | 3.128 | 1.857 | 0.7181 | 2.254 |
| 4) 4, 9, 14, 19 | 7.239 | 2.93 | 1.453 | 0.7057 | 2.806 |
| 5) 5, 10, 15, 20 | 7.77 | 2.799 | 1.619 | 0.4911 | 2.724 |
| 6) 6, 11, 16, 21 | 6.764 | 1.396 | 1.439 | 0.6159 | 2.263 |
| 7) 7, 12, 17, 22 | 6.491 | 1.45 | 1.133 | 0.6374 | 1.903 |

From the tables shown above, it can be seen that using four data points chosen in considerable distances, the sine curve can be successfully fitted out and the nerve tissue processes the largest coefficient a values. This demonstrates the ability to identify nerve based on four data points. In other embodiments, fewer data points may be used. Next, the same processing was performed on every pixel on the four chosen frames and a map of coefficient a was obtained as the output.

As the output a value maps of the chicken tissue model and cadaver model show, the nerve tissues stand out from the other tissues. With this technique, the rotating XPI system could be replaced by four pairs of crossed polarizers to provide four images to process. This may significantly reduce the amount of the sampling points needed and simplify the design of a XPI system. For example, such a system may be more easily integrated into a wearable device.

### Four-Angle Imaging Approach

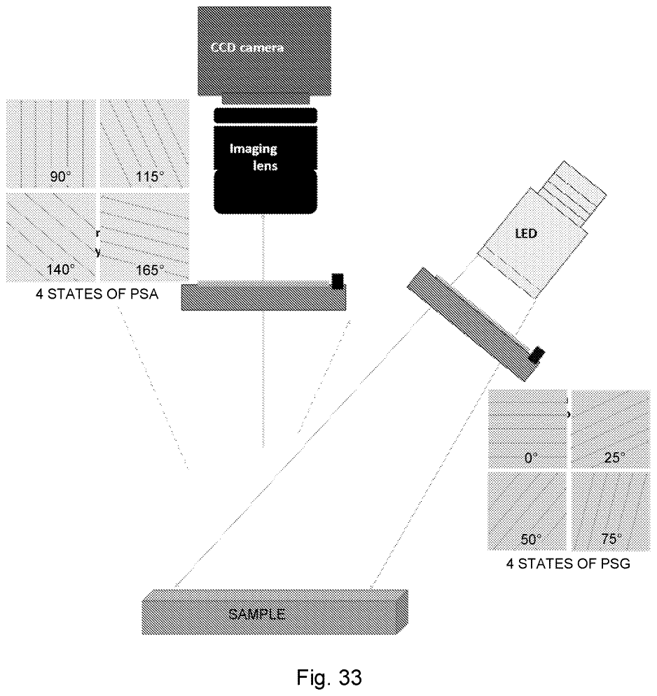

Using the curve fitting technique described above, a set of four XPI images at varying polarization angles (relative to the tissue) can provide sufficient data to obtain varying intensity useful to differentiate tissue types. Additional experiments were performed using a four-angle imaging approach (shown in FIG. 33) rather than a rotating XPI system. Instead of rotating the crossed polarizers and taking frames for many angles across the entire rotation, only four XPI images can be used. For example, the four images used in the experimental embodiment were: (1) PSG=0°, PSA=90°; (2) PSG=25°, PSA=115°; (3) PSG=50°, PSA=140°; and (4) PSG=75°, PSA=165°.

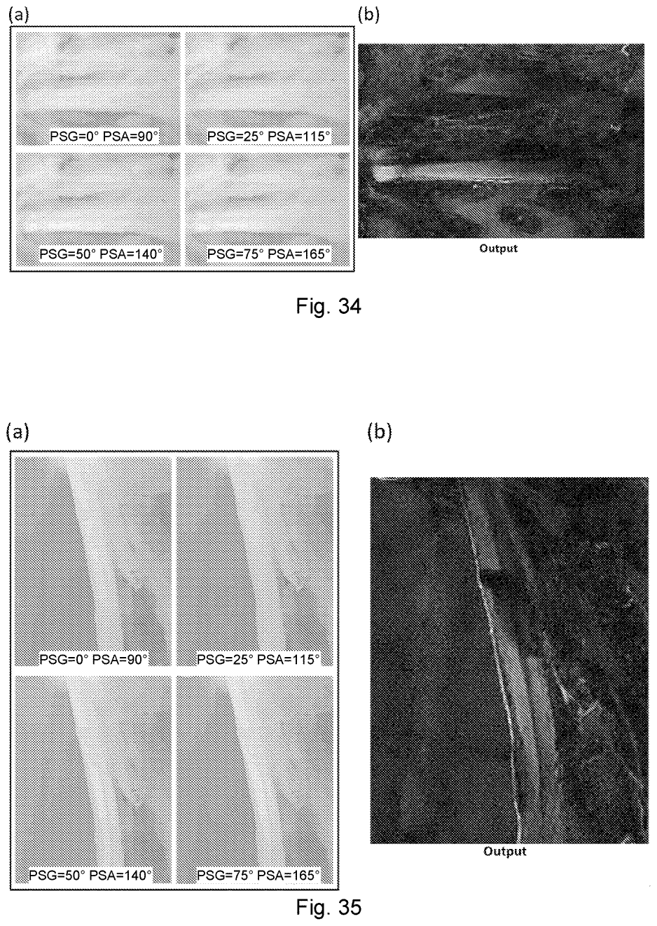

Sets of four XPI images were taken of two chicken thigh samples. Using only the recorded intensities of the four original images at four ‘equidistant’ angles, two output curve fitting results were calculated and shown in FIGS. 34 and 35, respectively. The resulting output images show the ability to differentiate nerve tissue using the reduced input data sets.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

What is claimed is:

1. A system for tissue identification, comprising:
a light source configured to illuminate a region of interest with polarized light, wherein the light source is further configured to provide polarized light at more than one polarization angle with respect to the region of interest;
an image sensor for acquiring images of the region of interest illuminated by the polarized light of the light source, the image sensor having a field of view;
at least one analyzer disposed within the field of view of the image sensor, wherein the analyzer is configured to have a polarization which maintains a non-zero angle relative to the polarization angle of the polarized light from the light source; and
a processor in electronic communication with the image sensor, wherein the processor is configured to:
obtain, from the image sensor, a series of images of the region of interest acquired using more than one polarization angle; and
identify a tissue within the region of interest based on an intensity of one or more pixels of the series of images varying according to the polarization angle.

2. The system of claim 1, wherein the light source is configured to provide polarized light at four polarization angles.

3. The system of claim 2, wherein the at least one analyzer comprises four analyzers, each analyzer configured to be orthogonal to a different polarization angle of the four polarization angles.

4. The system of claim 3, wherein the image sensor is configured to acquire four images, wherein each image is acquired by way of each of a corresponding one of the four analyzers.

5. The system of claim 1, wherein the light source is configured to provide polarized light at more than four polarization angles.

6. The system of claim 1, wherein the polarization of the light source is configured to rotate, and the analyzer is configured to rotate to maintain a non-zero angle relative to the polarization angle of the polarized light from the light source.

7. The system of claim 1, wherein the polarization of the polarized light is configured to rotate at least ¼ rotation during a sample period.

8. The system of claim 1, wherein the polarization of the polarized light is configured to rotate at least one full rotation during a sample period.

9. The system of claim 1, wherein the processor identifies the tissue as nerve tissue.

10. The system of claim 1, wherein the processor identifies the tissue as nerve, artery vein, fat, or muscle.

11. The system of claim 1, wherein the processor is configured to distinguish tissue types in the region of interest based on differences in the variability of intensity associated with tissue types.

12. The system of claim 1, wherein the polarized light and the analyzer are each linearly polarized.

13. The system of claim 1, wherein the polarization of the analyzer is configured to maintain orthogonality relative to the polarization angle of the polarized light.

14. The system of claim 1, wherein the image sensor acquires images in one or more limited spectral bands, and the processor identifies the tissue within the region of interest based on an intensity when illuminated by the one or more limited spectral bands.

15. The system of claim 14, wherein the light source is configured to provide light in one or more limited spectral bands.

16. The system of claim 15, wherein the light source is a broadband light source (e.g., a white light source) and an optical filter is disposed between the light source and the image sensor.

17. The system of claim 14, wherein the image sensor acquires images in a first limited spectral band for at least four different polarization angles of the polarized light.

18. The system of claim 17, wherein the image sensor acquires images in a second limited spectral band for at least a four different of the polarization of the polarized light.

19. The system of claim 1, wherein the processor is configured to identify the tissue by determining a frequency spectra time series of images obtained from the image sensor and determining pixels corresponding to the tissue based on a peak amplitude of a frequency corresponding to the tissue.

20. The system of claim 1, wherein the processor is configured to identify the tissue using lock-in signal detection to determine pixels corresponding to the tissue.

21. The system of claim 1, wherein the processor is further configured to generate an image mask based on image pixels corresponding to the identified tissue.

22. The system of claim 21, wherein the processor is further configured to alter an image or a series of images using the image mask.

23. The system of claim 22, wherein altering the image or series of images includes one or more of increasing an intensity of an unmasked portion of the image, drawing a boundary line around an unmasked portion of the image, changing a color of an unmasked portion of the image, and animating an unmasked portion of the image.

24. The system of claim 21, further comprising a projector in electronic communication with the processor, wherein the projector is configured to project, using the projector, a highlight on the region of interest based on the identified tissue.

25. The system of claim 21, further comprising a wearable display in electronic communication with the processor, wherein the wearable display is configured to provide augmented reality information to a wearer, and wherein the processor is further configured to provide, using the wearable display, a highlight overlaying the region of interest based on the identified tissue.

26. The system of claim 1, wherein the processor is further configured to identify the tissue as abnormal tissue.

27. The system of claim 26, wherein the processor identifies the tissue as abnormal based on the varying intensity.

28. The system of claim 1, wherein the processor is further configured to determine an orientation of the identified tissue.

29. A method for tissue identification, comprising:
illuminating a region of interest with a linearly-polarized light configured to have at least four polarization angles relative to the region of interest:
obtaining a series of images of the region of interest using an image sensor via an analyzer having a polarization configured to have at least four polarization angles which are orthogonal to the polarization angle of the linearly-polarized light; and
identifying a tissue within the region of interest based on an intensity of one or more pixels of the series of images varying according to the at least four polarization angles of the linearly-polarized light.

30. The method of claim 29, the tissue is identified as nerve tissue.

31. The method of claim 29, wherein the tissue is identified as nerve, artery vein, fat, or muscle.

32. The method of claim 29, wherein the tissue is identified by distinguishing tissue types in the region of interest based on differences in the variability of intensity associated with tissue types.

33. The method of claim 29, wherein the series of images is obtained in one or more limited spectral bands, and the tissue is identified based on an intensity when illuminated by the one or more limited spectral bands.

34. The method of claim 33, wherein the linearly-polarized light is configured to have one or more limited spectral bands.

35. The method of claim 33, wherein the images are acquired in a first limited spectral band for at least four different polarization angles of the linearly-polarized light.

36. The method of claim 35, wherein the images are acquired in a second limited spectral band for at least four different polarization angles of the linearly-polarized light.

37. The method of claim 29, wherein identifying the tissue comprises:
calculating a frequency spectra of the time series of images obtained from the image sensor; and
determining pixels corresponding to the tissue based on a peak amplitude of a frequency corresponding to the tissue.

38. The method of claim 29, wherein identifying the tissue comprises:
removing an offset from a set of time series values of a pixel to generate a centered signal for the pixel;
mixing a reference signal with the centered signal for the pixel;
shifting the phase of the reference signal and/or the centered signal to determine a phase-locked signal; and
calculating an average value of the phase-locked signal.

39. The method of claim 29, further comprising generating an image mask based on image pixels corresponding to the identified tissue.

40. The method of claim 39, further comprising altering an image or a series of images using the image mask.

41. The method of claim 40, wherein altering the image or series of images includes one or more of increasing an intensity of an unmasked portion of the image, drawing a boundary line around an unmasked portion of the image, changing a color of an unmasked portion of the image, and animating an unmasked portion of the image.

42. The method of claim 39, further comprising projecting a highlight on the region of interest based on the identified tissue.

43. The method of claim 39, further comprising providing augmented reality information to a wearable display.

44. The method of claim 43, wherein the augmented reality information is a highlight overlaying the region of interest based on the identified tissue.

45. The method of claim 29, further comprising identifying the tissue as abnormal tissue based on the varying intensity of the tissue.

46. The method of claim 29, further comprising determining an orientation of the identified tissue based on an intensity of the tissue corresponding to a polarization angle of the linearly-polarized light, and relative to the varying intensity of the tissue.
